(19) United States
(12) Patent Application Publication
(10) Pub. No.: US 2025/0177101 A1
(43) Pub. Date: Jun. 5, 2025

(54) INTRAORAL SCANNER INCLUDING PROJECTORS AND CAMERAS

(71) Applicant: Align Technology, Inc., San Jose, CA (US)

(72) Inventors: Nir Makmel, Tel-Aviv (IL); Yossef Y. Atiya, Modiin-Maccabim-Reut (IL)

(21) Appl. No.: 19/048,741

(22) Filed: Feb. 7, 2025

Related U.S. Application Data
(63) Continuation of application No. 18/605,466, filed on Mar. 14, 2024.
(60) Provisional application No. 63/453,004, filed on Mar. 17, 2023.
(60) Provisional application No. 63/470,022, filed on May 31, 2023.

(51) Int. Cl. A61C 9/00

(52) U.S. Cl. CPC A61C 9/006

(57) ABSTRACT

An intraoral scanner includes a rigid structure coupling with projectors and cameras. A field of view of each camera overlaps a field of view of a neighboring camera. The field of view of each camera overlaps a field of illumination of a neighboring projector. A projector is secured to a metallic frame at an opening of the metallic frame. Each projector includes: a light source to generate light; projector lenses to receive the light; projector lens frames to secure the projector lenses relative to the light source; and a pattern generating optical element to receive the light and generate a pattern of light along a projector axis onto an intraoral surface. Each camera includes: an image sensor to capture images along an optical axis that depict at least a portion of the pattern of light projected on the intraoral surface by the neighboring projector; and one or more camera lenses.

### RELATED APPLICATIONS

This application is a continuation application of U.S. patent application Ser. No. 18/605,466, filed Mar. 14, 2024, which claims benefit of U.S. Provisional Application No. 63/453,004, filed Mar. 17, 2023, and U.S. Provisional Application No. 63/470,022, filed May 31, 2023, the contents of which are hereby incorporated by reference in their entirety.

### TECHNICAL FIELD

Embodiments of the present disclosure relate to the field of dentistry and, in particular, to intraoral scanner projector alignment and fixing procedure.

### BACKGROUND

A dental site of a patient is to be measured accurately and studied carefully so that dental procedures can be performed.

### SUMMARY

Some example implementations of the present disclosure are summarized herein.

In a first implementation, a method includes: positioning projectors in a frame of an intraoral scanner; and responsive to the positioning of the projectors, soldering the projectors to a printed circuit board (PCB) of the intraoral scanner.

A second implementation may further extend the first implementation. In the second implementation, the intraoral scanner is configured to provide a plurality of intraoral scans of a dental site during an intraoral scanning session.

A third implementation may further extend the first or second implementations. In the third implementation, the plurality of intraoral scans are generated by projecting, via the projectors, a structured light comprising a plurality of features onto the dental site and capturing the plurality of features on the dental site.

A fourth implementation may further extend any of the first through third implementations. In the fourth implementation, the plurality of features comprise a plurality of spots.

A fifth implementation may further extend any of the first through fourth implementations. In the fifth implementation, the positioning of the projectors in the frame of the intraoral scanner comprises: positioning a first projector in a first orientation in the frame; positioning a second projector in a second orientation in the frame, wherein the second orientation is rotated about 3 to about 28 degrees from the first orientation; and positioning a third projector in a third orientation in the frame, wherein the third orientation is rotated about 3 to about 28 degrees from the second orientation.

A sixth implementation may further extend any of the first through fifth implementations. In the sixth implementation, the soldering of the projectors to the PCB comprises securing the first projector in the first orientation, the second projector in the second orientation, and the third projector in the third orientation in the intraoral scanner.

In a seventh implementation, a method includes: positioning a first projector in a first orientation in a frame of an intraoral scanner; positioning a second projector in a second orientation in the frame, wherein the second orientation is rotated about 3 to about 28 degrees from the first orientation; and positioning a third projector in a third orientation in the frame, wherein the third orientation is rotated about 3 to about 28 degrees from the second orientation; and securing the first projector in the first orientation, second projector in the second orientation, and the third projector in the third orientation in the intraoral scanner.

An eighth implementation may further extend the seventh implementation. In the eighth implementation, the first projector in the first orientation, the second projector in the second orientation, and the third projector in the third orientation reduces ambiguity of scanning of a dental site via the intraoral scanner.

A nineth implementation may further extend the seventh or eighth implementations. In the nineth implementation, the intraoral scanner is configured to provide a plurality of intraoral scans of a dental site during an intraoral scanning session.

A tenth implementation may further extend any of the seventh through nineth implementations. In the tenth implementation, the plurality of intraoral scans are generated by projecting, via a plurality of projectors, a structured light comprising a plurality of features onto the dental site and capturing the plurality of features on the dental site, the plurality of projectors comprising two or more of the first projector, the second projector, or the third projector.

An eleventh implementation may further extend any of the seventh through tenth implementations. In the eleventh implementation, the plurality of features comprise a plurality of spots.

A twelfth implementation may further extend any of the seventh through eleventh implementations. In the twelfth implementation, the securing of the first projector in the first orientation, the second projector in the second orientation, and the third projector in the third orientation comprises soldering the first projector, the second projector, and the third projector to a printed circuit board (PCB) of the intraoral scanner.

In a thirteenth implementation, an intraoral scanner includes: a projector comprising: a diode; a lens disposed at a distal end of the diode; a bushing disposed around a first distal end of the diode; a flange disposed around a second distal end of the diode; and a sleeve disposed around a central portion of the diode.

A fourteenth implementation may further extend the thirteenth implementation. In the fourteenth implementation, the projector is configured to perform structured light scanning.

A fifteenth implementation may further extend the thirteenth or fourteenth implementations. In the fifteenth implementation: the sleeve is welded to the bushing; and the flange and the sleeve are welded to each other.

A sixteenth implementation may further extend any of the thirteenth through fifteenth implementations. In the sixteenth implementation, the intraoral scanner further includes a lens guard attached to the bushing, wherein the lens guard is configured to prevent the lens from dislodging from the projector.

In a seventeenth implementation, an intraoral scanner includes: a projector comprising: a diode; a lens disposed at a distal end of the diode; a bushing disposed around a first distal end of the diode; and a lens guard attached to the bushing, wherein the lens guard is configured to prevent the lens from dislodging from the projector.

An eighteenth implementation may further extend the seventeenth implementation. In the eighteenth implementation, the intraoral scanner further includes a sleeve disposed around a central portion of the diode.

A nineteenth implementation may further extend the seventeenth or eighteenth implementations. In the nineteenth implementation, the sleeve is welded to the bushing.

A twentieth implementation may further extend any of the seventeenth through nineteenth implementations. In the twentieth implementation, the intraoral scanner further includes a flange disposed around a second distal end of the diode.

A twenty-first implementation may further extend any of the seventeenth through twentieth implementations. In the twenty-first implementation, the flange and the sleeve are welded to each other.

A twenty-second implementation may further extend any of the seventeenth through twenty-first implementations. In the twenty-second implementation, the projector is configured to perform structured light scanning.

### BRIEF DESCRIPTION OF THE DRAWINGS

Embodiments of the present disclosure are illustrated by way of example, and not by way of limitation, in the figures of the accompanying drawings.

FIG. 1 illustrates a system for performing intraoral scanning and/or generating a virtual three-dimensional model of a dental site, according to certain embodiments.

FIG. 2A is a schematic illustration of a handheld intraoral scanner with a plurality of cameras disposed within a probe at a distal end of the intraoral scanner, according to certain embodiments.

FIGS. 2B-2C include schematic illustrations of positioning configurations for cameras and structured light projectors of an intraoral scanner, according to certain embodiments.

FIG. 2D is a chart depicting a plurality of different configurations for the position of structured light projectors and cameras in a probe of an intraoral scanner, according to certain embodiments.

FIGS. 3A-N illustrate components of intraoral scanners, according to certain embodiments.

FIGS. 4A-H illustrate components of projectors of intraoral scanners, according to certain embodiments.

FIGS. 5A-B are flow charts of methods associated with projectors of intraoral scanners, according to certain embodiments.

FIG. 6 illustrates a block diagram of an example computing device, according to certain embodiments.

### DETAILED DESCRIPTION

Described herein are methods and systems associated with alignment and fixing of projectors in an intraoral scanner (e.g., projector alignment and fixing procedure).

A dental site of a patient is to be measured accurately and studied carefully so that dental procedures can be performed. For example, in prosthodontic procedures designed to implant a dental prosthesis in the oral cavity, the dental site at which the prosthesis is to be implanted in many cases should be measured accurately and studied carefully, so that a prosthesis such as a crown, denture or bridge, for example, can be properly designed and dimensioned to fit in place. A good fit enables mechanical stresses to be properly transmitted between the prosthesis and the jaw, and to prevent infection of the gums via the interface between the prosthesis and the dental site, for example. Some procedures also call for removable prosthetics to be fabricated to replace one or more missing teeth, such as a partial or full denture, in which case the surface contours of the areas where the teeth are missing need to be reproduced accurately so that the resulting prosthetic fits over the edentulous region with even pressure on the soft tissues.

In some practices, the dental site is prepared by a dental practitioner, and a positive physical model of the dental site is constructed using known methods. Alternatively, the dental site may be scanned to provide 3D data of the dental site. In either case, the virtual or real model of the dental site is sent to the dental lab, which manufactures the prosthesis based on the model. However, if the model is deficient or undefined in certain areas, or if the preparation was not optimally configured for receiving the prosthesis, the design of the prosthesis may be less than optimal. For example, if the insertion path implied by the preparation for a closely-fitting coping would result in the prosthesis colliding with adjacent teeth, the coping geometry has to be altered to avoid the collision, which may result in the coping design being less optimal. Further, if the area of the preparation containing a finish line lacks definition, it may not be possible to properly determine the finish line and thus the lower edge of the coping may not be properly designed. Indeed, in some circumstances, the model is rejected and the dental practitioner then re-scans the dental site, or reworks the preparation, so that a suitable prosthesis may be produced.

In orthodontic procedures it can be important to provide a model of one or both jaws. Where such orthodontic procedures are designed virtually, a virtual model of the oral cavity is also beneficial. Such a virtual model may be obtained by scanning the oral cavity directly, or by producing a physical model of the dentition, and then scanning the model with a suitable scanner.

Thus, in both prosthodontic and orthodontic procedures, obtaining a three-dimensional (3D) model of a dental site in the oral cavity is an initial procedure that is performed. When the 3D model is a virtual model, the more complete and accurate the scans of the dental site are, the higher the quality of the virtual model, and thus the greater the ability to design an optimal prosthesis or orthodontic treatment appliance(s).

A scanner may have multiple projectors and multiple cameras. Each projector may project a pattern of light on a dental site in the field of view of at least one camera. The cameras capture images of the patterns of light on the dental site. The images are used to generate a 3D model of the dental site. Some conventional scanners have ambiguity of which projected patterns are of which projector, which causes ambiguity and lower accuracy in the resulting 3D models. Some conventional scanners are manufactured with misaligned projectors which causes lower accuracy in the resulting 3D models. Some conventional scanners have projectors that become misaligned over time due to being thermally sensitive, creeping under stresses, shrinkage forces, etc. which causes lower accuracy in resulting 3D models. Lower accuracy in 3D models results in incorrectly designed dental devices (e.g., dental appliances, dental aligners, prosthetics, etc.), repair or replacement of scanners, and waste of material, additional time and processing, etc.

The devices, systems, and methods of the present disclosure overcome some or all of these challenges.

In some embodiments, a method includes positioning projectors in a frame of an intraoral scanner and, responsive to the positioning of the projectors, soldering the projectors to the printed circuit board (PCB) of the intraoral scanner. The intraoral scanner may be configured to provide intraoral scans of a dental site during an intraoral scanning session. The intraoral scans may be generated by projecting, via the projectors, a structured light including features (e.g., spots, a projected pattern) onto the dental site and capturing the features on the dental site.

In some embodiments, a method includes positioning a first projector in a first orientation in a frame of an intraoral scanner, positioning a second projector in a second orientation in the frame, wherein the second orientation is rotated about 3 to about 28 degrees from the first orientation, positioning a third projector in a third orientation in the frame, wherein the third orientation is rotated about 3 to about 28 degrees from the second orientation, and securing the first projector in the first orientation, second projector in the second orientation, and the third projector in the third orientation in the intraoral scanner. In some embodiments, the first projector in the first orientation, second projector in the second orientation, and the third projector in the third orientation reduces ambiguity of scanning of a dental site via the intraoral scanner.

In some embodiments, an intraoral scanner includes a projector that includes a diode, a lens disposed at a distal end of the diode, a bushing disposed around the first distal end of the diode, a flange disposed around a second distal end of the diode, and a sleeve disposed around a central portion of the diode. The projector is configured to perform structured light scanning. In some embodiments, the sleeve is welded to the bushing. The flange and the sleeve may be welded to each other.

In some embodiments, an intraoral scanner includes a projector that includes a diode, a lens disposed at a distal end of the diode, a bushing disposed around the first distal end of the diode, and a lens guard attached to the bushing. The lens guard is configured to prevent the lens from dislodging from the projector.

The devices systems, and methods of the present disclosure have advantages over conventional systems. The intraoral scanner of the present disclosure has projectors that are more accurately aligned and maintain alignment better than conventional systems. This results in less repair and replacement of scanners and less waste of material. The intraoral scanner of the present disclosure can estimate a 3D surface with a higher degree of accuracy than conventional systems. This results in less time and processing and more accurately designed dental devices compared to conventional systems.

Various embodiments are described herein. These various embodiments may be implemented as stand-alone solutions and/or may be combined. Accordingly, references to an embodiment, one embodiment, or some embodiments may refer to the same embodiment and/or to different embodiments. Some embodiments are discussed herein with reference to intraoral scans and intraoral images. However, embodiments described with reference to intraoral scans also apply to lab scans or model/impression scans. A lab scan or model/impression scan may include one or more images of a dental site or of a model or impression of a dental site, which may or may not include height maps, and which may or may not include intraoral two-dimensional (2D) images (e.g., 2D color images).

In some embodiments, the present disclosure describes intraoral scanners including projectors and cameras. In some embodiments, the projectors and/or cameras of the present disclosure may be part of a system that is not an intraoral scanner.

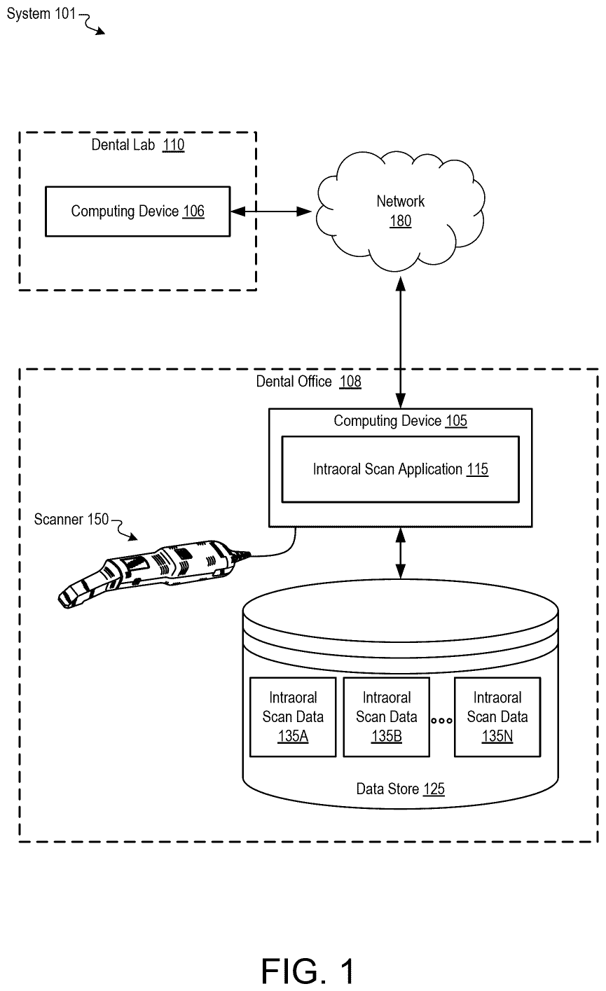

FIG. 1 illustrates a system 101 for performing intraoral scanning and/or generating a three-dimensional (3D) surface and/or a virtual three-dimensional model of a dental site, according to certain embodiments. System 101 includes a scanner 150. The scanner 150 may be the intraoral scanner of the present disclosure (e.g., includes projectors of the present disclosure).

System 101 includes a dental office 108 and optionally one or more dental lab 110. The dental office 108 and the dental lab 110 each include a computing device 105, 106, where the computing devices 105, 106 may be connected to one another via a network 180. The network 180 may be a local area network (LAN), a public wide area network (WAN) (e.g., the Internet), a private WAN (e.g., an intranet), or a combination thereof.

Computing device 105 may be coupled to one or more intraoral scanner 150 (also referred to as a scanner) and/or a data store 125 via a wired or wireless connection. In some embodiments, multiple scanners 150 in dental office 108 wirelessly connect to computing device 105. In some embodiments, scanner 150 is wirelessly connected to computing device 105 via a direct wireless connection. In some embodiments, scanner 150 is wirelessly connected to computing device 105 via a wireless network. In some embodiments, the wireless network is a Wi-Fi network. In some embodiments, the wireless network is a Bluetooth network, a Zigbee network, or some other wireless network. In some embodiments, the wireless network is a wireless mesh network, examples of which include a Wi-Fi mesh network, a Zigbee mesh network, and so on. In an example, computing device 105 may be physically connected to one or more wireless access points and/or wireless routers (e.g., Wi-Fi access points/routers). Intraoral scanner 150 may include a wireless module such as a Wi-Fi module, and via the wireless module may join the wireless network via the wireless access point/router.

Computing device 106 may also be connected to a data store (not shown). The data stores may be local data stores and/or remote data stores. Computing device 105 and computing device 106 may each include one or more processing devices, memory, secondary storage, one or more input devices (e.g., such as a keyboard, mouse, tablet, touchscreen, microphone, camera, and so on), one or more output devices (e.g., a display, printer, touchscreen, speakers, etc.), and/or other hardware components.

In embodiments, scanner 150 includes an inertial measurement unit (IMU). The IMU may include an accelerometer, a gyroscope, a magnetometer, a pressure sensor, and/or other type of sensor. For example, scanner 150 may include one or more micro-electromechanical system (MEMS) IMU. The IMU may generate inertial measurement data (also referred to as movement data), including acceleration data, rotation data, and so on.

Computing device 105 and/or data store 125 may be located at dental office 108 (as shown), at dental lab 110, or at one or more other locations such as a server farm that provides a cloud computing service. Computing device 105 and/or data store 125 may connect to components that are at a same or a different location from computing device 105 (e.g., components at a second location that is remote from the dental office 108, such as a server farm that provides a cloud computing service). For example, computing device 105 may be connected to a remote server, where some operations of intraoral scan application 115 are performed on computing device 105 and some operations of intraoral scan application 115 are performed on the remote server.

Some additional computing devices may be physically connected to the computing device 105 via a wired connection. Some additional computing devices may be wirelessly connected to computing device 105 via a wireless connection, which may be a direct wireless connection or a wireless connection via a wireless network. In embodiments, one or more additional computing devices may be mobile computing devices such as laptops, notebook computers, tablet computers, mobile phones, portable game consoles, and so on. In embodiments, one or more additional computing devices may be traditionally stationary computing devices, such as desktop computers, set top boxes, game consoles, and so on. The additional computing devices may act as thin clients to the computing device 105. In some embodiments, the additional computing devices access computing device 105 using remote desktop protocol (RDP). In some embodiments, the additional computing devices access computing device 105 using virtual network control (VNC). Some additional computing devices may be passive clients that do not have control over computing device 105 and that receive a visualization of a user interface of intraoral scan application 115. In some embodiments, one or more additional computing devices may operate in a master mode and computing device 105 may operate in a slave mode.

Intraoral scanner 150 may include a probe (e.g., a handheld probe) for optically capturing three-dimensional structures. The intraoral scanner 150 may be used to perform an intraoral scan of a patient's oral cavity. An intraoral scan application 115 running on computing device 105 may communicate with the scanner 150 to effectuate the intraoral scan. A result of the intraoral scan may be intraoral scan data 135A, 135B through 135N that may include one or more sets of intraoral scans and/or sets of intraoral 2D images. Each intraoral scan may include a 3D image or point cloud that may include depth information (e.g., a height map) of a portion of a dental site. In embodiments, intraoral scans include x, y, and z information.

Intraoral scan data 135A-N may also include color 2D images and/or images of wavelengths (e.g., near-infrared (NIRI) images, infrared images, ultraviolet images, etc.) of a dental site in embodiments. In embodiments, intraoral scanner 150 alternates between generation of 3D intraoral scans and one or more types of 2D intraoral images (e.g., color images, NIRI images, etc.) during scanning. For example, one or more 2D color images may be generated between generation of a fourth and fifth intraoral scan by outputting white light and capturing reflections of the white light using multiple cameras.

Intraoral scanner 150 may include multiple different cameras (e.g., each of which may include one or more image sensors) that generate 2D images (e.g., 2D color images) of different regions of a patient's dental arch concurrently. These 2D images may be stitched together to form a single 2D image representation of a larger field of view that includes a combination of the fields of view of the multiple cameras. Intraoral 2D images may include 2D color images, 2D infrared or near-infrared (NIRI) images, and/or 2D images generated under other specific lighting conditions (e.g., 2D ultraviolet images). The 2D images may be used by a user of the intraoral scanner to determine where the scanning face of the intraoral scanner is directed and/or to determine other information about a dental site being scanned.

The scanner 150 may transmit the intraoral scan data 135A, 135B through 135N to the computing device 105. Computing device 105 may store the intraoral scan data 135A-135N in data store 125.

According to an example, a user (e.g., a practitioner) may subject a patient to intraoral scanning. In doing so, the user may apply scanner 150 to one or more patient intraoral locations. The scanning may be divided into one or more segments (also referred to as roles). As an example, the segments may include a lower dental arch of the patient, an upper dental arch of the patient, one or more preparation teeth of the patient (e.g., teeth of the patient to which a dental device such as a crown or other dental prosthetic will be applied), one or more teeth which are contacts of preparation teeth (e.g., teeth not themselves subject to a dental device but which are located next to one or more such teeth or which interface with one or more such teeth upon mouth closure), and/or patient bite (e.g., scanning performed with closure of the patient's mouth with the scan being directed towards an interface area of the patient's upper and lower teeth). Via such scanner application, the scanner 150 may provide intraoral scan data 135A-N to computing device 105. The intraoral scan data 135A-N may be provided in the form of intraoral scan data sets, each of which may include 2D intraoral images (e.g., color 2D images) and/or 3D intraoral scans of particular teeth and/or regions of a dental site. In some embodiments, separate intraoral scan data sets are created for the maxillary arch, for the mandibular arch, for a patient bite, and/or for each preparation tooth. Alternatively, a single large intraoral scan data set is generated (e.g., for a mandibular and/or maxillary arch). Intraoral scans may be provided from the scanner 150 to the computing device 105 in the form of one or more points (e.g., one or more pixels and/or groups of pixels). For instance, the scanner 150 may provide an intraoral scan as one or more point clouds. The intraoral scans may each include height information (e.g., a height map that indicates a depth for each pixel).

The manner in which the oral cavity of a patient is to be scanned may depend on the procedure to be applied thereto. For example, if an upper or lower denture is to be created, then a full scan of the mandibular or maxillary edentulous arches may be performed. In contrast, if a bridge is to be created, then just a portion of a total arch may be scanned which includes an edentulous region, the neighboring preparation teeth (e.g., abutment teeth) and the opposing arch and dentition. Alternatively, full scans of upper and/or lower dental arches may be performed if a bridge is to be created.

By way of non-limiting example, dental procedures may be broadly divided into prosthodontic (restorative) and orthodontic procedures, and then further subdivided into specific forms of these procedures. Additionally, dental procedures may include identification and treatment of gum disease, sleep apnea, and intraoral conditions. The term prosthodontic procedure refers, inter alia, to any procedure involving the oral cavity and directed to the design, manufacture, or installation of a dental prosthesis at a dental site within the oral cavity (dental site), or a real or virtual model thereof, or directed to the design and preparation of the dental site to receive such a prosthesis. A prosthesis may include any restoration such as crowns, veneers, inlays, onlays, implants and bridges, for example, and any other artificial partial or complete denture. The term orthodontic procedure refers, inter alia, to any procedure involving the oral cavity and directed to the design, manufacture, or installation of orthodontic elements at a dental site within the oral cavity, or a real or virtual model thereof, or directed to the design and preparation of the dental site to receive such orthodontic elements. These elements may be appliances including but not limited to brackets and wires, retainers, clear aligners, or functional appliances.

In embodiments, intraoral scanning may be performed on a patient's oral cavity during a visitation of dental office 108. The intraoral scanning may be performed, for example, as part of a semi-annual or annual dental health checkup. The intraoral scanning may also be performed before, during and/or after one or more dental treatments, such as orthodontic treatment and/or prosthodontic treatment. The intraoral scanning may be a full or partial scan of the upper and/or lower dental arches and may be performed to gather information for performing dental diagnostics, to generate a treatment plan, to determine progress of a treatment plan, and/or for other purposes. The dental information (intraoral scan data 135A-N) generated from the intraoral scanning may include 3D scan data, 2D color images, NIRI and/or infrared images, and/or ultraviolet images, of all or a portion of the upper jaw and/or lower jaw. The intraoral scan data 135A-N may further include one or more intraoral scans showing a relationship of the upper dental arch to the lower dental arch. These intraoral scans may be usable to determine a patient bite and/or to determine occlusal contact information for the patient. The patient bite may include determined relationships between teeth in the upper dental arch and teeth in the lower dental arch.

For many prosthodontic procedures (e.g., to create a crown, bridge, veneer, etc.), an existing tooth of a patient is ground down to a stump. The ground tooth is referred to herein as a preparation tooth, or simply a preparation. The preparation tooth has a margin line (also referred to as a finish line), which is a border between a natural (unground) portion of the preparation tooth and the prepared (ground) portion of the preparation tooth. The preparation tooth is typically created so that a crown or other prosthesis can be mounted or seated on the preparation tooth. In many instances, the margin line of the preparation tooth is sub-gingival (below the gum line).

Intraoral scanners may work by moving the scanner 150 inside a patient's mouth to capture all viewpoints of one or more tooth. During scanning, the scanner 150 is calculating distances to solid surfaces in some embodiments. These distances may be recorded as images called ‘height maps’ or as point clouds in some embodiments. Each scan (e.g., optionally height map or point cloud) is overlapped algorithmically, or ‘stitched,’ with the previous set of scans to generate a growing 3D surface. As such, each scan is associated with a rotation in space, or a projection, to how it fits into the 3D surface.

During intraoral scanning, intraoral scan application 115 may register and stitch together two or more intraoral scans generated thus far from the intraoral scan session to generate a growing 3D surface. In some embodiments, performing registration includes capturing 3D data of various points of a surface in multiple scans, and registering the scans by computing transformations between the scans. One or more 3D surfaces may be generated based on the registered and stitched together intraoral scans during the intraoral scanning. The one or more 3D surfaces may be output to a display so that a doctor or technician can view their scan progress thus far. As each new intraoral scan is captured and registered to previous intraoral scans and/or a 3D surface, the one or more 3D surfaces may be updated, and the updated 3D surface(s) may be output to the display. A view of the 3D surface(s) may be periodically or continuously updated according to one or more viewing modes of the intraoral scan application. In one viewing mode, the 3D surface may be continuously updated such that an orientation of the 3D surface that is displayed aligns with a field of view of the intraoral scanner (e.g., so that a portion of the 3D surface that is based on a most recently generated intraoral scan is approximately centered on the display or on a window of the display) and a user sees what the intraoral scanner sees. In one viewing mode, a position and orientation of the 3D surface is static, and an image of the intraoral scanner is optionally shown to move relative to the stationary 3D surface.

Intraoral scan application 115 may generate one or more 3D surfaces from intraoral scans and may display the 3D surfaces to a user (e.g., a doctor) via a graphical user interface (GUI) during intraoral scanning. In embodiments, separate 3D surfaces are generated for the upper jaw and the lower jaw. This process may be performed in real time or near-real time to provide an updated view of the captured 3D surfaces during the intraoral scanning process. As scans are received, these scans may be registered and stitched to a 3D surface. Quality scores may be determined for various regions of the 3D surface based on one or more criteria as discussed in detail below. The quality scores may be continuously or periodically updated as information is added from further intraoral scans. As the quality scores gradually change, a visualization of the regions may change in accordance with the changes in the quality scores, enabling a user to have real time or near real time feedback on surface quality during scanning. Additionally, or alternatively, as scans are received the scanning process may be monitored to determine if a user is having trouble scanning any regions of a dental site (e.g., of the upper or lower dental arch). If a determination is made that a user is having trouble scanning a region of the dental site, then one or more remedial actions may be performed and/or one or more suggestions may be provided. Additionally, or alternatively, as scanning is being performed a zoom setting for displaying the 3D surface(s) may be dynamically determined based on one or more criteria, such as a velocity of the scanner and/or of a point of focus of the scanner. In embodiments, a user may select to enable or disable automatic zoom and/or automatic suggestions via the GUI. For example, the user may input a request for scanning assistance, which may cause automatic zoom and/or scanning suggestions to be enabled. These and other operations may be performed during scanning to improve a quality of the 3D surface(s), to speed up scanning, to help a user in trouble areas, and so on.

When a scan session or a portion of a scan session associated with a particular scanning role (e.g., upper jaw role, lower jaw role, bite role, etc.) is complete (e.g., all scans for an dental site or dental site have been captured), intraoral scan application 115 may generate a virtual 3D model of one or more scanned dental sites (e.g., of an upper jaw and a lower jaw). The final 3D model may be a set of 3D points and their connections with each other (i.e., a mesh). To generate the virtual 3D model, intraoral scan application 115 may register and stitch together the intraoral scans generated from the intraoral scan session that are associated with a particular scanning role. The registration performed at this stage may be more accurate than the registration performed during the capturing of the intraoral scans and may take more time to complete than the registration performed during the capturing of the intraoral scans. In some embodiments, performing scan registration includes capturing 3D data of various points of a surface in multiple scans, and registering the scans by computing transformations between the scans. The 3D data may be projected into a 3D space of a 3D model to form a portion of the 3D model. The intraoral scans may be integrated into a common reference frame by applying appropriate transformations to points of each registered scan and projecting each scan into the 3D space.

In some embodiments, registration is performed for adjacent or overlapping intraoral scans (e.g., each successive frame of an intraoral video). Registration algorithms are carried out to register two adjacent or overlapping intraoral scans and/or to register an intraoral scan with a 3D model, which essentially involves determination of the transformations which align one scan with the other scan and/or with the 3D model. Registration may involve identifying multiple points in each scan (e.g., point clouds) of a scan pair (or of a scan and the 3D model), surface fitting to the points, and using local searches around points to match points of the two scans (or of the scan and the 3D model). For example, intraoral scan application 115 may match points of one scan with the closest points interpolated on the surface of another scan, and iteratively minimize the distance between matched points. Other registration techniques may also be used.

Intraoral scan application 115 may repeat registration for all intraoral scans of a sequence of intraoral scans to obtain transformations for each intraoral scan, to register each intraoral scan with previous intraoral scan(s) and/or with a common reference frame (e.g., with the 3D model). Intraoral scan application 115 may integrate intraoral scans into a single virtual 3D model by applying the appropriate determined transformations to each of the intraoral scans. Each transformation may include rotations about one to three axes and translations within one to three planes.

Intraoral scan application 115 may generate one or more 3D models from intraoral scans and may display the 3D models to a user (e.g., a doctor) via a graphical user interface (GUI). The 3D models can then be checked visually by the doctor. The doctor can virtually manipulate the 3D models via the user interface with respect to up to six degrees of freedom (i.e., translated and/or rotated with respect to one or more of three mutually orthogonal axes) using suitable user controls (hardware and/or virtual) to enable viewing of the 3D model from any desired direction.

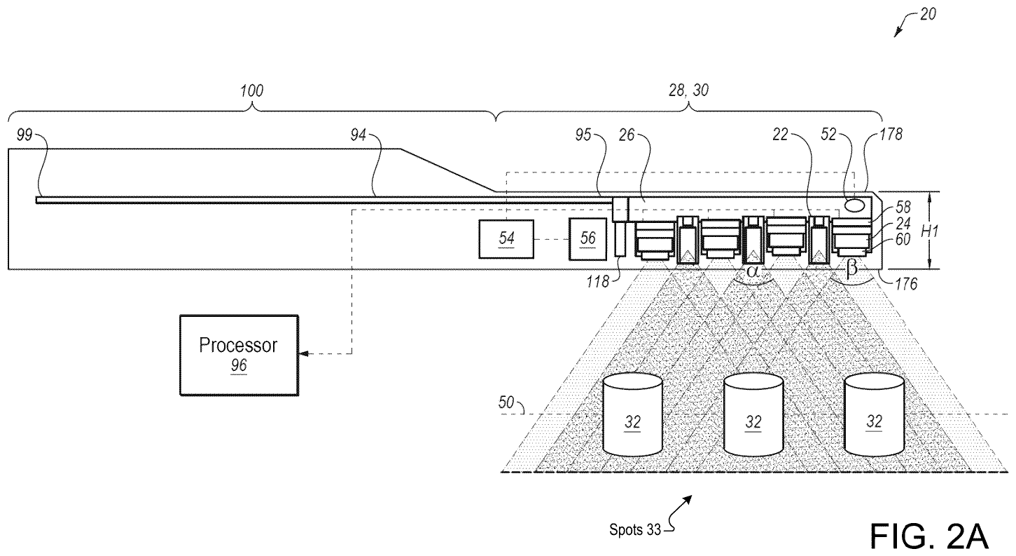

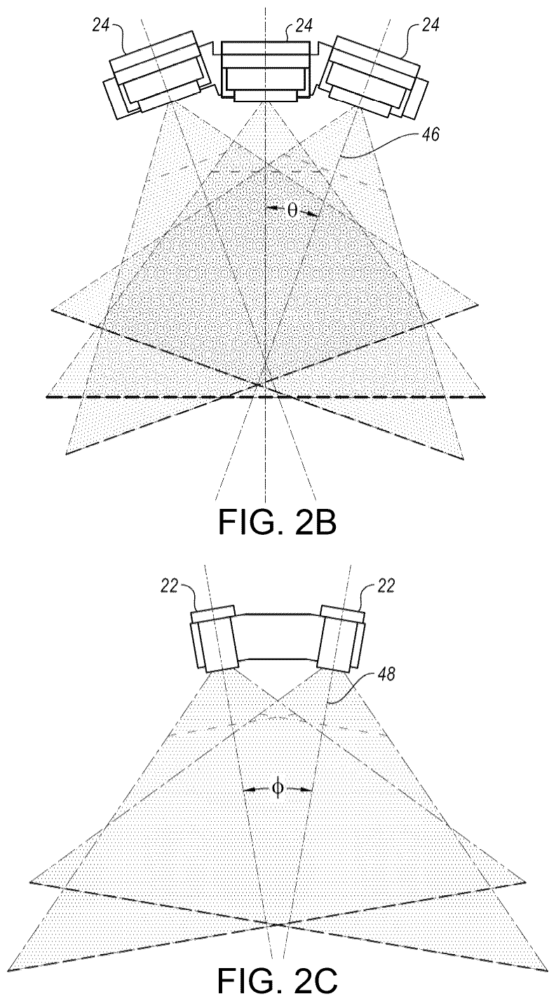

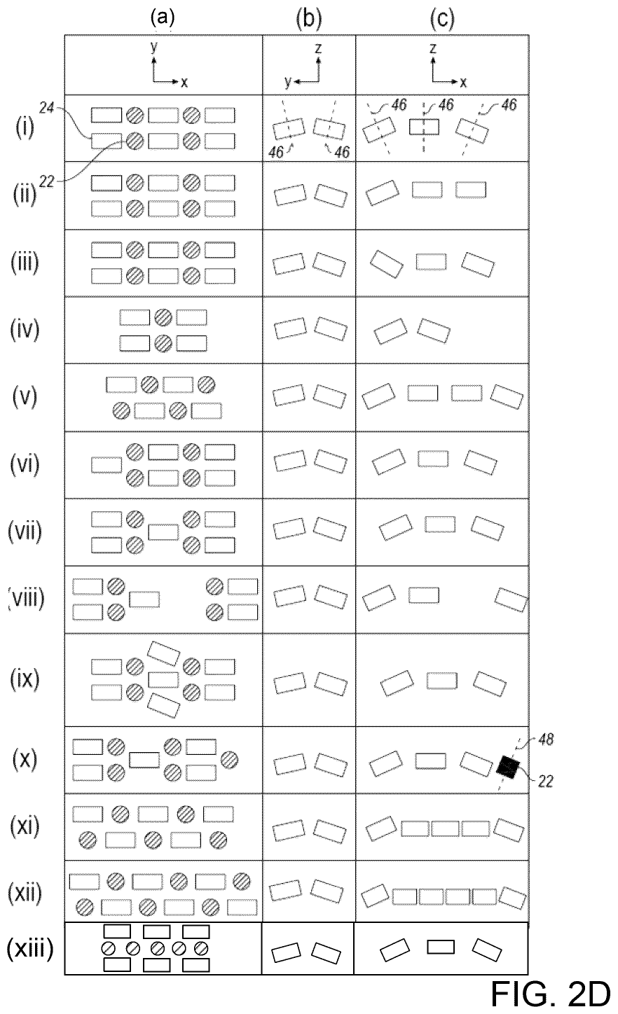

Reference is now made to FIG. 2A, which is a schematic illustration of an intraoral scanner 20 including an elongate handheld wand, according to certain embodiments. The intraoral scanner 20 may correspond to intraoral scanner 150 of FIG. 1 in some embodiments. Intraoral scanner 20 may be the intraoral scanner of the present disclosure (e.g., includes projectors of the present disclosure).

Intraoral scanner 20 includes a plurality of structured light projectors 22 (e.g., projectors) and a plurality of cameras 24 that are coupled to a rigid structure 26 disposed within a probe 28 at a distal end 30 of the intraoral scanner 20. In some applications, during an intraoral scanning procedure, probe 28 is inserted into the oral cavity of a subject or patient.

For some applications, structured light projectors 22 are positioned within probe 28 such that each structured light projector 22 faces an object 32 outside of intraoral scanner 20 that is placed in its field of illumination, as opposed to positioning the structured light projectors in a proximal end of the handheld wand and illuminating the object by reflection of light off a mirror and subsequently onto the object. Alternatively, the structured light projectors may be disposed at a proximal end of the handheld wand. Similarly, for some applications, cameras 24 are positioned within probe 28 such that each camera 24 faces an object 32 outside of intraoral scanner 20 that is placed in its field of view, as opposed to positioning the cameras in a proximal end of the intraoral scanner and viewing the object by reflection of light off a mirror and into the camera. This positioning of the projectors and the cameras within probe 28 enables the scanner to have an overall large field of view while maintaining a low-profile probe. Alternatively, the cameras may be disposed in a proximal end of the handheld wand.

In some applications, cameras 24 each have a large field of view β (beta) of at least 45 degrees, e.g., at least 70 degrees, e.g., at least 80 degrees, e.g., 85 degrees. In some applications, the field of view may be less than 120 degrees, e.g., less than 100 degrees, e.g., less than 90 degrees. In some embodiments, a field of view β (beta) for each camera is between 80 and 90 degrees, which may be particularly useful because it provided a good balance among pixel size, field of view and camera overlap, optical quality, and cost. Cameras 24 may include an image sensor 58 and objective optics 60 including one or more lenses. To enable close focus imaging, cameras 24 may focus on an object focal plane 50 that is located between 1 mm and 30 mm, e.g., between 4 mm and 24 mm, e.g., between 5 mm and 11 mm, e.g., 9 mm-10 mm, from the lens that is farthest from the sensor. In some applications, cameras 24 may capture images at a frame rate of at least 30 frames per second, e.g., at a frame of at least 75 frames per second, e.g., at least 100 frames per second. In some applications, the frame rate may be less than 200 frames per second.

A large field of view achieved by combining the respective fields of view of all the cameras may improve accuracy due to reduced amount of image stitching errors, especially in edentulous regions, where the gum surface is smooth and there may be fewer clear high resolution 3D features. Having a larger field of view enables large smooth features, such as the overall curve of the tooth, to appear in each image frame, which improves the accuracy of stitching respective surfaces obtained from multiple such image frames.

Similarly, structured light projectors 22 may each have a large field of illumination a (alpha) of at least 45 degrees, e.g., at least 70 degrees. In some applications, field of illumination a (alpha) may be less than 120 degrees, e.g., than 100 degrees.

For some applications, to improve image capture, each camera 24 has a plurality of discrete preset focus positions, in each focus position the camera focusing on a respective object focal plane 50. Each of cameras 24 may include an autofocus actuator that selects a focus position from the discrete preset focus positions to improve a given image capture. Additionally or alternatively, each camera 24 includes an optical aperture phase mask that extends a depth of focus of the camera, such that images formed by each camera are maintained focused over all object distances located between 1 mm and 30 mm, e.g., between 4 mm and 24 mm, e.g., between 5 mm and 11 mm, e.g., 9 mm-10 mm, from the lens that is farthest from the sensor.

In some applications, structured light projectors 22 and cameras 24 are coupled to rigid structure 26 in a closely packed and/or alternating fashion, such that (a) a substantial part of each camera's field of view overlaps the field of view of neighboring cameras, and (b) a substantial part of each camera's field of view overlaps the field of illumination of neighboring projectors. Optionally, at least 20%, e.g., at least 50%, e.g., at least 75% of the projected pattern of light are in the field of view of at least one of the cameras at an object focal plane 50 that is located at least 4 mm from the lens that is farthest from the sensor. Due to different possible configurations of the projectors and cameras, some of the projected pattern may never be seen in the field of view of any of the cameras, and some of the projected pattern may be blocked from view by object 32 as the scanner is moved around during a scan.

Rigid structure 26 may be a non-flexible structure to which structured light projectors 22 and cameras 24 are coupled so as to provide structural stability to the optics within probe 28. Coupling all the projectors and all the cameras to a common rigid structure helps maintain geometric integrity of the optics of each structured light projector 22 and each camera 24 under varying ambient conditions, e.g., under mechanical stress as may be induced by the subject's mouth. Additionally, rigid structure 26 helps maintain stable structural integrity and positioning of structured light projectors 22 and cameras 24 with respect to each other.

Reference is now made to FIGS. 2B-2C, which include schematic illustrations of a positioning configuration for cameras 24 and structured light projectors 22 respectively, according to certain embodiments. The cameras 24 and/or structured light projectors 22 may be of the intraoral scanner of the present disclosure.

For some applications, to improve the overall field of view and field of illumination of the intraoral scanner 20, cameras 24 and structured light projectors 22 are positioned such that they do not all face the same direction. For some applications, such as is shown in FIG. 2B, a plurality of cameras 24 are coupled to rigid structure 26 such that an angle θ (theta) between two respective optical axes 46 of at least two cameras 24 is 90 degrees or less, e.g., 35 degrees or less. Similarly, for some applications, such as is shown in FIG. 2C, a plurality of structured light projectors 22 are coupled to rigid structure 26 such that an angle q (phi) between two respective optical axes 48 of at least two structured light projectors 22 is 90 degrees or less, e.g., 35 degrees or less.

Reference is now made to FIG. 2D, which is a chart depicting a plurality of different configurations for the position of structured light projectors 22 and cameras 24 in probe 28, according to certain embodiments. The cameras 24 and/or structured light projectors 22 may be of the intraoral scanner of the present disclosure.

Structured light projectors 22 are represented in FIG. 2D by circles and cameras 24 are represented in FIG. 2D by rectangles. It is noted that rectangles are used to represent the cameras, since typically, each image sensor 58 and the field of view β (beta) of each camera 24 have aspect ratios of 1:2. Column (a) of FIG. 2D shows a bird's eye view of the various configurations of structured light projectors 22 and cameras 24. The x-axis as labeled in the first row of column (a) corresponds to a central longitudinal axis of probe 28. Column (b) shows a side view of cameras 24 from the various configurations as viewed from a line of sight that is coaxial with the central longitudinal axis of probe 28 and substantially parallel to a viewing axis of the intraoral scanner. Similar to as shown in FIG. 2B, column (b) of FIG. 2D shows cameras 24 positioned so as to have optical axes 46 at an angle of 90 degrees or less, e.g., 35 degrees or less, with respect to each other. Column (c) shows a side view of cameras 24 of the various configurations as viewed from a line of sight that is perpendicular to the central longitudinal axis of probe 28.

Typically, the distal-most (toward the positive x-direction in FIG. 2D) and proximal-most (toward the negative x-direction in FIG. 2D) cameras 24 are positioned such that their optical axes 46 are slightly turned inwards, e.g., at an angle of 90 degrees or less, e.g., 35 degrees or less, with respect to the next closest camera 24. The camera(s) 24 that are more centrally positioned, i.e., not the distal-most camera 24 nor proximal-most camera 24, are positioned so as to face directly out of the probe, their optical axes 46 being substantially perpendicular to the central longitudinal axis of probe 28. It is noted that in row (xi) a projector 22 is positioned in the distal-most position of probe 28, and as such the optical axis 48 of that projector 22 points inwards, allowing a larger number of spots 33 projected from that particular projector 22 to be seen by more cameras 24.

In embodiments, the number of structured light projectors 22 in probe 28 may range from two, e.g., as shown in row (iv) of FIG. 2D, to six, e.g., as shown in row (xii). Typically, the number of cameras 24 in probe 28 may range from four, e.g., as shown in rows (iv) and (v), to seven, e.g., as shown in row (ix). It is noted that the various configurations shown in FIG. 2D are by way of example and not limitation, and that the scope of the present disclosure includes additional configurations not shown. For example, the scope of the present disclosure includes fewer or more than five projectors 22 positioned in probe 28 and fewer or more than seven cameras positioned in probe 28.

In an example application, an apparatus for intraoral scanning (e.g., an intraoral scanner 150) includes an elongate handheld wand including a probe at a distal end of the elongate handheld wand, at least two light projectors disposed within the probe, and at least four cameras disposed within the probe. Each light projector may include at least one light source configured to generate light when activated, and a pattern generating optical element that is configured to generate a pattern of light when the light is transmitted through the pattern generating optical element. Each of the at least four cameras may include a camera sensor (also referred to as an image sensor) and one or more lenses, wherein each of the at least four cameras is configured to capture a plurality of images that depict at least a portion of the projected pattern of light on an intraoral surface. A majority of the at least two light projectors and the at least four cameras may be arranged in at least two rows that are each approximately parallel to a longitudinal axis of the probe, the at least two rows including at least a first row and a second row.

In a further application, a distal-most camera along the longitudinal axis and a proximal-most camera along the longitudinal axis of the at least four cameras are positioned such that their optical axes are at an angle of 90 degrees or less with respect to each other from a line of sight that is perpendicular to the longitudinal axis. Cameras in the first row and cameras in the second row may be positioned such that optical axes of the cameras in the first row are at an angle of 90 degrees or less with respect to optical axes of the cameras in the second row from a line of sight that is coaxial with the longitudinal axis of the probe. A remainder of the at least four cameras other than the distal-most camera and the proximal-most camera have optical axes that are substantially parallel to the longitudinal axis of the probe. Each of the at least two rows may include an alternating sequence of light projectors and cameras.

In a further application, the at least four cameras include at least five cameras, the at least two light projectors include at least five light projectors, a proximal-most component in the first row is a light projector, and a proximal-most component in the second row is a camera.

In a further application, the distal-most camera along the longitudinal axis and the proximal-most camera along the longitudinal axis are positioned such that their optical axes are at an angle of 35 degrees or less with respect to each other from the line of sight that is perpendicular to the longitudinal axis. The cameras in the first row and the cameras in the second row may be positioned such that the optical axes of the cameras in the first row are at an angle of 35 degrees or less with respect to the optical axes of the cameras in the second row from the line of sight that is coaxial with the longitudinal axis of the probe.

In a further application, the at least four cameras may have a combined field of view of 25-45 mm along the longitudinal axis and a field of view of 20-40 mm along a z-axis corresponding to distance from the probe.

Returning to FIG. 2A, for some applications, there is at least one uniform light projector 118 (which may be an unstructured light projector that projects light across a range of wavelengths) coupled to rigid structure 26. Uniform light projector 118 may transmit white light onto object 32 being scanned. At least one camera, e.g., one of cameras 24, captures two-dimensional color images of object 32 using illumination from uniform light projector 118.

Processor 96 may run a surface reconstruction algorithm that may use detected patterns (e.g., dot patterns) projected onto object 32 to generate a 3D surface of the object 32. In some embodiments, the processor 96 may combine at least one 3D scan captured using illumination from structured light projectors 22 with a plurality of intraoral 2D images captured using illumination from uniform light projector 118 in order to generate a digital three-dimensional image of the intraoral three-dimensional surface. Using a combination of structured light and uniform illumination enhances the overall capture of the intraoral scanner and may help reduce the number of options that processor 96 needs to consider when running a correspondence algorithm used to detect depth values for object 32. In some embodiments, the intraoral scanner and correspondence algorithm described in U.S. application Ser. No. 16/446,181, filed Jun. 19, 2019, is used. U.S. application Ser. No. 16/446,181, filed Jun. 19, 2019, is incorporated by reference herein in its entirety. In embodiments, processor 96 may be a processor of computing device 105 of FIG. 1. Alternatively, processor 96 may be a processor integrated into the intraoral scanner 20.

For some applications, all data points taken at a specific time are used as a rigid point cloud, and multiple such point clouds are captured at a frame rate of over 10 captures per second. The plurality of point clouds is then stitched together using a registration algorithm, e.g., iterative closest point (ICP), to create a dense point cloud. A surface reconstruction algorithm may then be used to generate a representation of the surface of object 32.

For some applications, at least one temperature sensor 52 is coupled to rigid structure 26 and measures a temperature of rigid structure 26. Temperature control circuitry 54 disposed within intraoral scanner 20 (a) receives data from temperature sensor 52 indicative of the temperature of rigid structure 26 and (b) activates a temperature control unit 56 in response to the received data. Temperature control unit 56, e.g., a PID controller, keeps probe 28 at a desired temperature (e.g., between 35 and 43 degrees Celsius, between 37 and 41 degrees Celsius, etc.). Keeping probe 28 above 35 degrees Celsius, e.g., above 37 degrees Celsius, reduces fogging of the glass surface of intraoral scanner 20, through which structured light projectors 22 project and cameras 24 view, as probe 28 enters the intraoral cavity, which is typically around or above 37 degrees Celsius. Keeping probe 28 below 43 degrees, e.g., below 41 degrees Celsius, prevents discomfort or pain.

In some embodiments, heat may be drawn out of the probe 28 via a heat conducting element 94, e.g., a heat pipe, that is disposed within intraoral scanner 20, such that a distal end 95 of heat conducting element 94 is in contact with rigid structure 26 and a proximal end 99 is in contact with a proximal end 100 of intraoral scanner 20. Heat is thereby transferred from rigid structure 26 to proximal end 100 of intraoral scanner 20. Alternatively, or additionally, a fan disposed in a handle region 174 of intraoral scanner 20 may be used to draw heat out of probe 28.

FIGS. 2A-2D illustrate one type of intraoral scanner that can be used for embodiments of the present disclosure. However, embodiments are not limited to the illustrated type of intraoral scanner. In some embodiments, intraoral scanner 150 corresponds to the intraoral scanner described in U.S. application Ser. No. 16/910,042, filed Jun. 23, 2020 and entitled “Intraoral 3D Scanner Employing Multiple Miniature Cameras and Multiple Miniature Pattern Projectors”, which is incorporated by reference herein. In some embodiments, intraoral scanner 150 corresponds to the intraoral scanner described in U.S. application Ser. No. 16/446,181, filed Jun. 19, 2019 and entitled “Intraoral 3D Scanner Employing Multiple Miniature Cameras and Multiple Miniature Pattern Projectors”, which is incorporated by reference herein.

In some embodiments an intraoral scanner that performs confocal focusing to determine depth information may be used. Such an intraoral scanner may include a light source and/or illumination module that emits light (e.g., a focused light beam or array of focused light beams). The light passes through a polarizer and through a unidirectional mirror or beam splitter (e.g., a polarizing beam splitter) that passes the light. The light may pass through a pattern before or after the beam splitter to cause the light to become patterned light. Along an optical path of the light after the unidirectional mirror or beam splitter are optics, which may include one or more lens groups. Any of the lens groups may include only a single lens or multiple lenses. One of the lens groups may include at least one moving lens.

The light may pass through an endoscopic probing member, which may include a rigid, light-transmitting medium, which may be a hollow object defining within it a light transmission path or an object made of a light transmitting material, e.g., a glass body or tube. In some embodiments, the endoscopic probing member includes a prism such as a folding prism. At its end, the endoscopic probing member may include a mirror of the kind ensuring a total internal reflection. Thus, the mirror may direct the array of light beams towards a teeth segment or other object. The endoscope probing member thus emits light, which optionally passes through one or more windows and then impinges on to surfaces of intraoral objects.

The light may include an array of light beams arranged in an X-Y plane, in a Cartesian frame, propagating along a Z axis, which corresponds to an imaging axis or viewing axis of the intraoral scanner. Responsive to the surface on which the incident light beams hits being an uneven surface, illuminated spots may be displaced from one another along the Z axis, at different (Xᵢ, Yᵢ) locations. Thus, while a spot at one location may be in focus of the confocal focusing optics, spots at other locations may be out-of-focus. Therefore, the light intensity of returned light beams of the focused spots will be at its peak, while the light intensity at other spots will be off peak. Thus, for each illuminated spot, multiple measurements of light intensity are made at different positions along the Z-axis. For each of such (Xᵢ, Yᵢ) location, the derivative of the intensity over distance (Z) may be made, with the Zᵢ yielding maximum derivative, Z₀, being the in-focus distance.

The light reflects off intraoral objects and passes back through windows (if they are present), reflects off of the mirror, passes through the optical system, and is reflected by the beam splitter onto a detector. The detector is an image sensor having a matrix of sensing elements each representing a pixel of the scan or image. In some embodiments, the detector is a charge coupled device (CCD) sensor. In some embodiments, the detector is a complementary metal-oxide semiconductor (CMOS) type image sensor. Other types of image sensors may also be used for detector. In some embodiments, the detector detects light intensity at each pixel, which may be used to compute height or depth.

Alternatively, in some embodiments an intraoral scanner that uses stereo imaging is used to determine depth information.

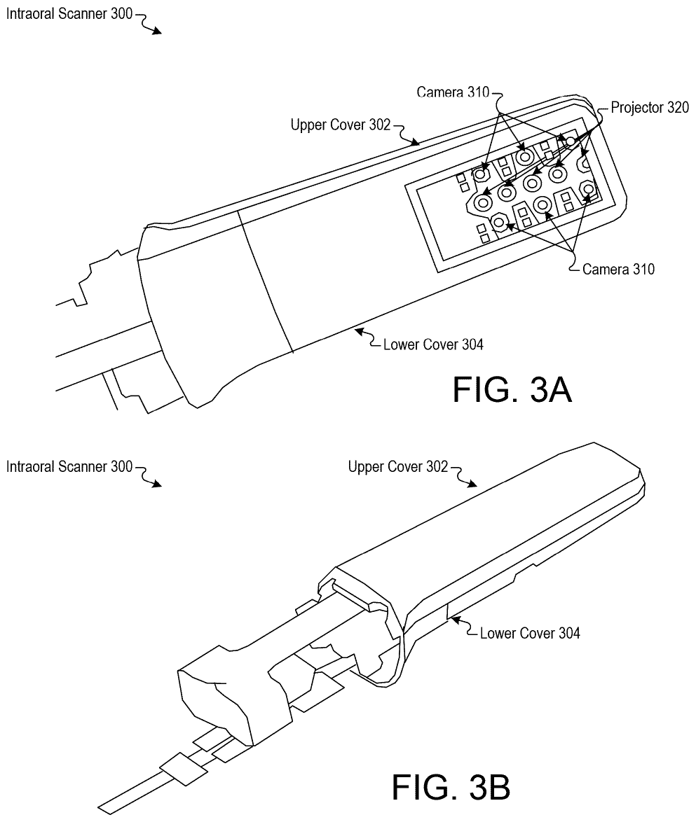

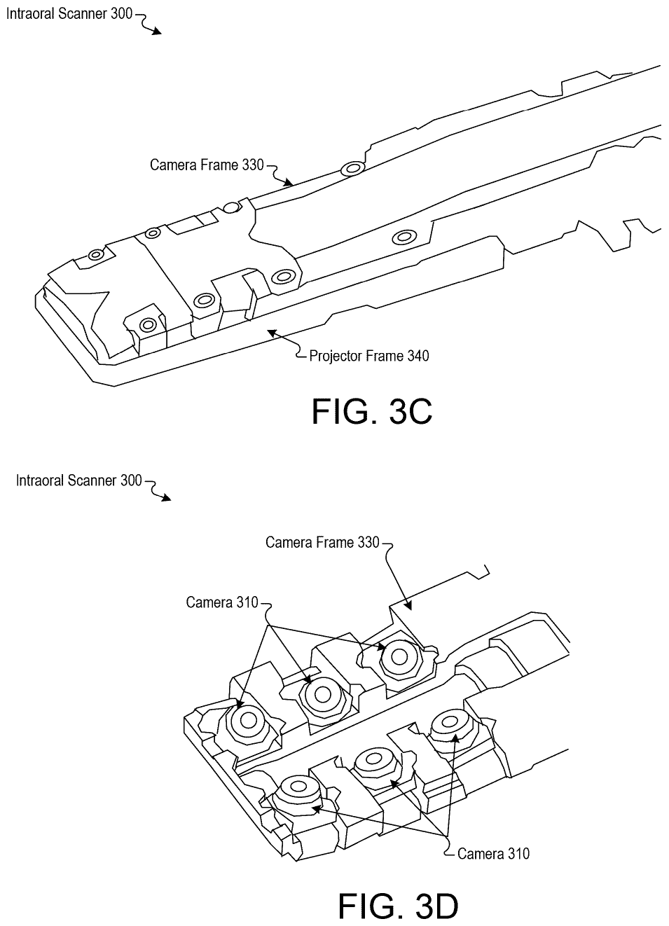

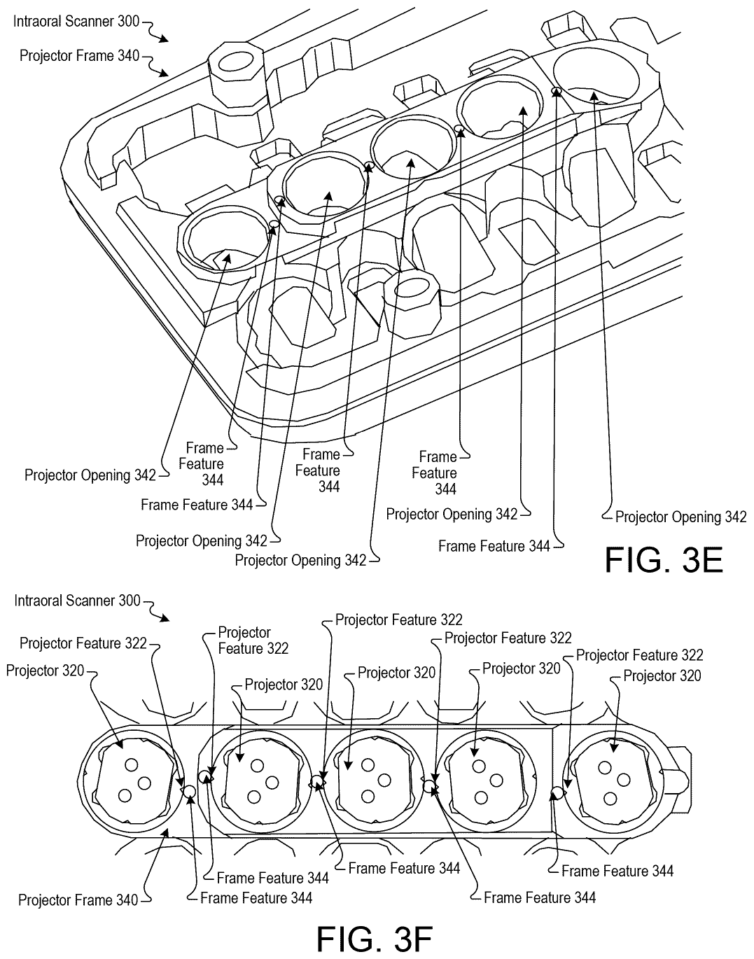

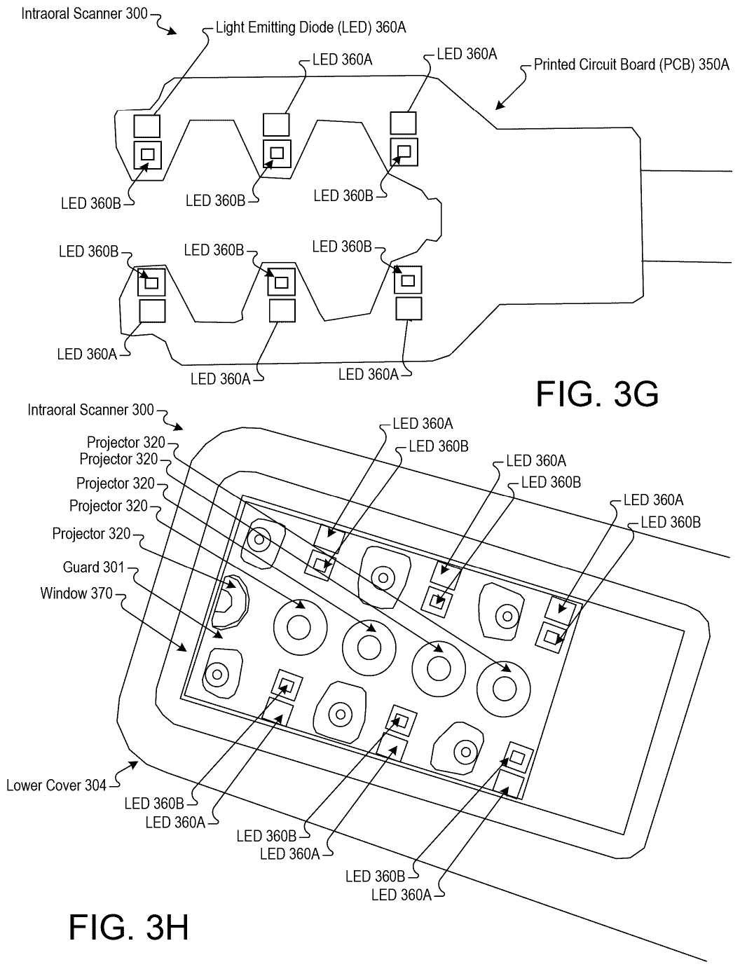

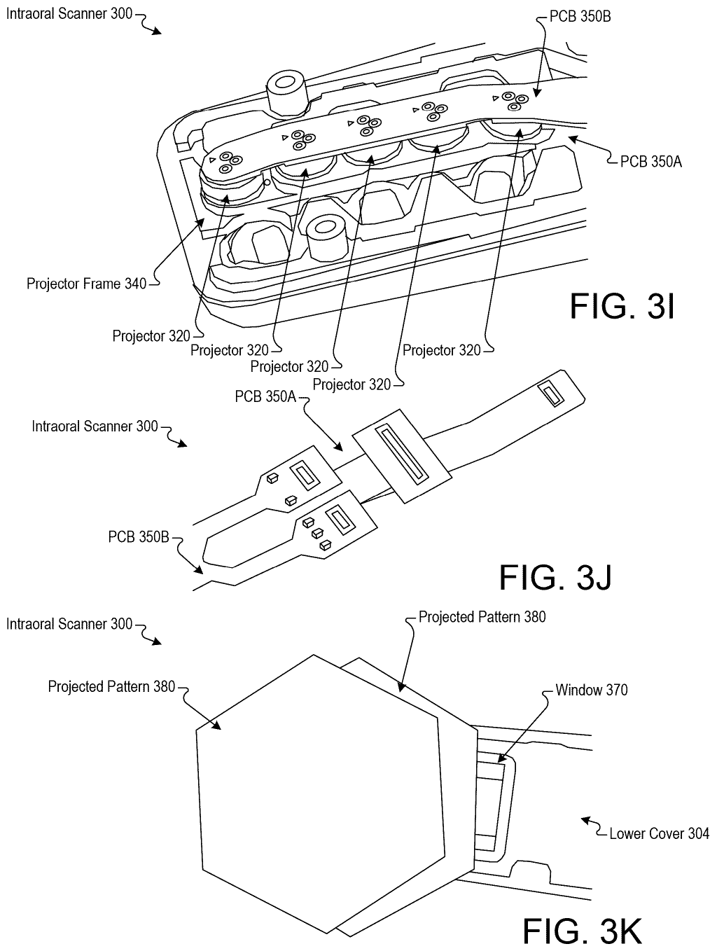

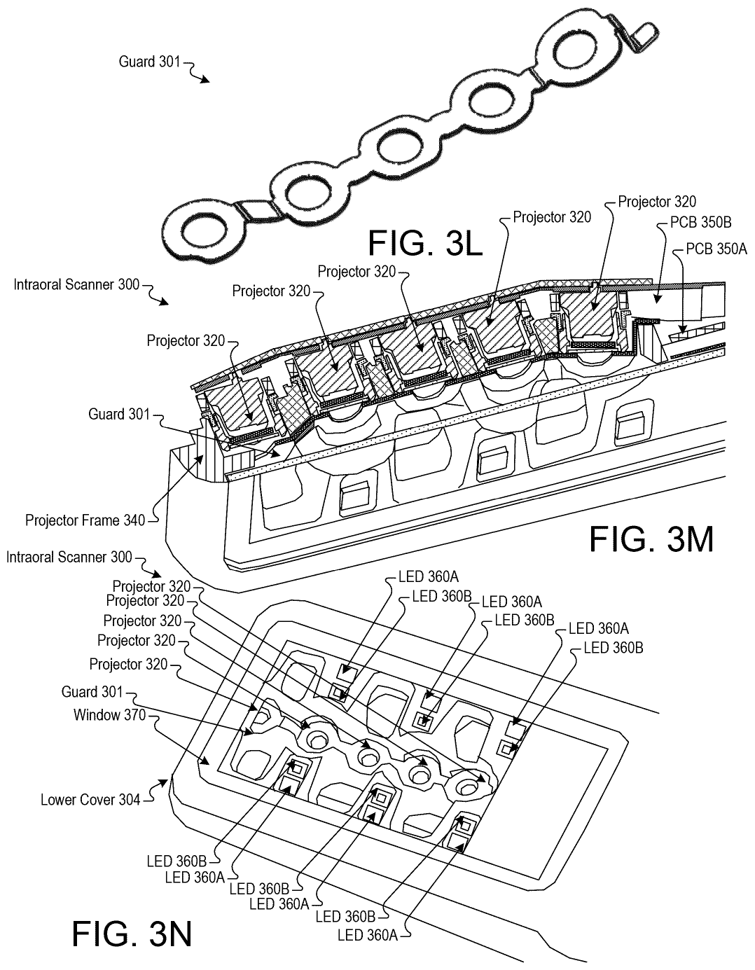

FIGS. 3A-N illustrate components of intraoral scanners 300, according to certain embodiments. In some embodiments, intraoral scanners 300 of one or more of FIGS. 3A-N include similar or the same functionality, components, materials, and/or the like as scanner 150 of FIG. 1 and/or intraoral scanner 20 of FIGS. 2A-D.

FIG. 3A illustrates a bottom perspective view of the intraoral scanner 300. FIG. 3B illustrates a top perspective view of the intraoral scanner 300. The tip of the intraoral scanner 300 may have an upper cover 302 (e.g., upper plastic cover) and a lower cover 304 (e.g., lower plastic cover).

The intraoral scanner 300 may be a wand that is connected to a computation station. The tip of the intraoral scanner 300 may be inserted into the oral cavity of a person while scanning procedure is performed. A disposable sleeve may be placed over the tip of the intraoral scanner 300 prior to being inserted in the oral cavity. The tip of the intraoral scanner 300 may be part of an assembly that is separate from the rest of the wand. Once the assembly of the tip of the intraoral scanner 300 is completed, the tip can be added to the wand. The tip may connect to the wand via mechanical interfaces (e.g., screws, springs, bolts, fasteners) and electrical connections (e.g., connect to internal portion of scanner). The tip of the intraoral scanner 300 may be a standalone item that is later integrated with the rest of the intraoral scanner 300 (e.g., endpiece, scanner). The intraoral scanner 300 may use multi-structured light to create a 3D model. In some embodiments, the intraoral scanner 300 has six cameras inside square holes formed by the intraoral scanner 300. The intraoral scanner 300 may have five projectors that are disposed between the cameras (e.g., a row of three cameras disposed proximate a first side of the intraoral scanner 300, a row of three cameras disposed proximate a second side of the intraoral scanner 300, and a row of five projectors disposed between the two rows of cameras. In some embodiments, three of the projectors are blue illuminations and two of the projectors are green illumination. The multiple cameras and multiple projectors may be used to provide sufficient data for 3D construction (e.g., 3D imaging) of the dental site.

FIG. 3C illustrates the camera frame 330 and the projector frame 340 secured to each other in the intraoral scanner 300. FIG. 3D illustrates cameras 310 disposed in the camera frame 330 of the intraoral scanner 300.

Inside the upper cover 302 and lower cover 304, the intraoral scanner 300 may have a camera frame 330 (e.g., upper piece, camera holder) configured to secure cameras 310 and a projector frame 340 (e.g., lower piece, projector holder) configured to secure projectors 320. The camera frame 330 and the projector frame 340 may be separate components. The camera frame 330 may be used for heating, thermal management, and/or holding the cameras 310 in different directions. The cameras 310 may be tilted to cover the field of view of the scan area.

FIG. 3E illustrates a projector frame 340 of the intraoral scanner 300. FIG. 3F illustrates projectors 320 disposed in the projector frame 340 of the intraoral scanner 300.

The projector frame 340 may be metallic (e.g., aluminum). The projector frame 340 may secure projectors 320 (e.g., five projectors 320) in the middle of the projector frame 340. The projector frame 340 may have a dedicated projector opening 342 (e.g., hole) for each projector 320. Each projector 320 may be positioned in a corresponding projector opening 342 (e.g., hole) of the projector frame 340. Each projector 320 may be attached to the projector frame 340 at a corresponding projector opening 342 (e.g., hole) via adhesive and then the projector frame 340 with projectors 320 may be cured inside an oven so that the projectors 320 and projector frame 340 (e.g., aluminum frame) become one piece.

The projectors 340 may fit inside the projector openings 342 without an air gap. Heat may be dissipated from the projectors 320 through the projector frame 340.

The projector frame 340 forms frame features 344 (e.g., recesses, holes, markings, drilled recesses) indicating where corresponding projector features 322 (e.g., notches, markings) are to align. For example, a projector 320 may be rotated until the frame feature 344 and the projector feature 322 align. In some embodiments, each of the projectors 320 (e.g., aligned via the projector feature 322 and frame feature 344) are at different orientations than each other. In some embodiments, the orientation of each projector 320 is rotated about 3 to about 28 degrees from the orientation of each of the other projectors 320. In some embodiments, each of the projectors 320 are disposed in a different orientation (e.g., different angle in theta-z around projector main axis) in the intraoral scanner 300. For example, a first projector 320 may be oriented at about-10 degrees, a second projector 320 may be oriented at about-5 degrees, a third projector 320 may be oriented at about 0 degrees, a fourth projector 320 may be oriented at about +5 degrees, and a fifth projector 320 may be oriented at about +10 degrees. The orientation of the projectors 320 may form a bowl shape. The orientation of each of the projectors 320 may have a tolerance of +/−2 degrees.

FIG. 3G illustrates a printed circuit board (PCB) 350A of the intraoral scanner 300. FIG. 3H illustrates a window 370 of the intraoral scanner 300.

PCB 350A may include light emitting diodes (LEDs) 360. LEDs 360A may be white LEDs. LEDs 360B may be infrared LEDs. LEDs 360A may be used for color reconstruction. The window 370 may be an electrically heated window on a front of the intraoral scanner 300 for defogging purposes. In some embodiments, intraoral scanner one or more guards 301 are used to project one or more projectors 320. In some examples, a common guard 301 protects all projectors 320 (e.g., five projectors 320) together at the frame assembly level. In some examples, a multiple guards 301 are used (e.g., one guard 301 to secure one projector 320).

FIG. 3I illustrates a first distal portion of PCB 350A and first distal portion of PCB 350B attached to the projectors 320 disposed in the projector frame 340 of the intraoral scanner 300. FIG. 3J illustrates a second distal portion of the PCB 350A and PCB 350B.

The PCB 350B is secured (e.g., soldered) to the projectors 320 subsequent to the projectors being positioned in the projector frame 340. In some embodiments, the projectors 320 are positioned in the projector frame 340 with adhesive (e.g., frictionally secured in projector frame 340), the projectors are soldered to the PCB 350B, and then the adhesive between the projector frame 340 and the projectors 320 is cured in an oven (e.g., projectors 320 are fixed into place in the projector frame 340). Each projector 320 may be fixed to a designated opening by using thermally conductive adhesive, which also improves the thermal connection of the projector 320 to the projector frame 340.

In some embodiments, three of the projectors 320 are based on blue laser illumination and two of the projectors 320 are based on green laser illumination. Each projector 320 may have three legs, where two legs (e.g., anode and cathode) are soldered to the PCB 350B and the third leg is ground.

The PCB 350B may form holes that align with the corresponding orientation of each of the projectors 320 (e.g., −10, −5, 0, 5, and 10 degrees). The soldering of the PCB 350B to the projectors 320 maintains the projectors 320 in the correct orientations until the adhesive is cured.

A second distal portion of PCB 350B may have a first leg that is coupled to the projectors 320 that perform blue illumination and may have a second leg that is coupled to the projectors 320 that perform green illumination. The second distal portions of PCB 350A and PCB 350B may be used to electrically couple to the LEDs 360 and projectors 320.

FIG. 3K illustrates projected patterns 380 that are projected by the projectors 320 of the intraoral scanner 300. Each of the projectors 320 are at different orientations (e.g., angles, rotations) so that the projected patterns 380 are not parallel. This prevents ambiguity of which projected pattern 380 is from which projector 320. Each of the projectors 320 are rotated relative to each other (e.g., to generate angle differentiation between projected patterns 380) to avoid ambiguity of projected patterns 380.

FIGS. 3L-N illustrate guard 301 of intraoral scanners 300, according to certain embodiments. Guard 301 may be a common guard 301 that secures multiple projectors 320. FIG. 3L illustrates a guard 301 (e.g., common guard 301). FIG. 3M illustrates a cross-sectional perspective view of intraoral scanner 300 that includes a guard 301 securing projectors 320. FIG. 3N illustrates a perspective view of an intraoral scanner 300 that includes a guard 301 securing projectors 320. The guard 301 (e.g., common guard) may be attached to a predesigned space in the projector frame 340 via friction fit (e.g., by snapping without adhesive). The guard 301 may have a thickness of about 0.1 to about 0.3 mm (e.g., about 0.2 mm). The guard 301 may be metal (e.g., stainless steel alloy, chromium-nickel stainless steel, stainless steel 302, etc.).

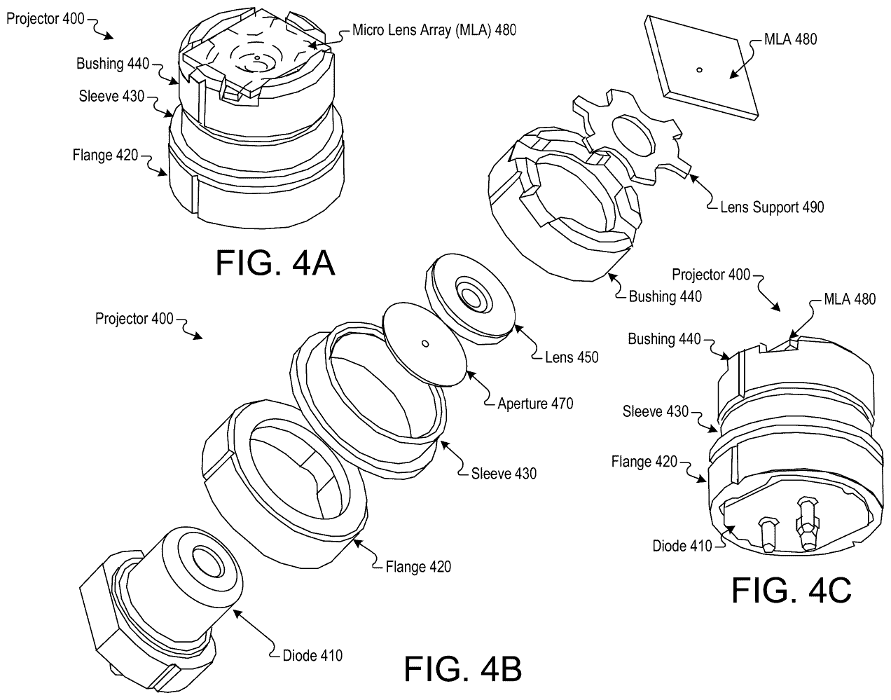

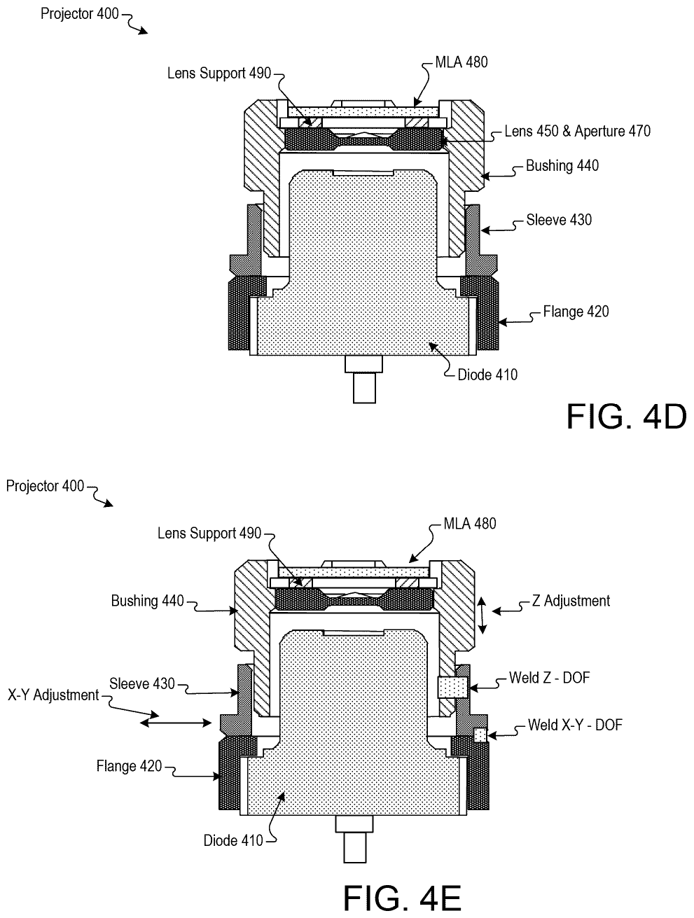

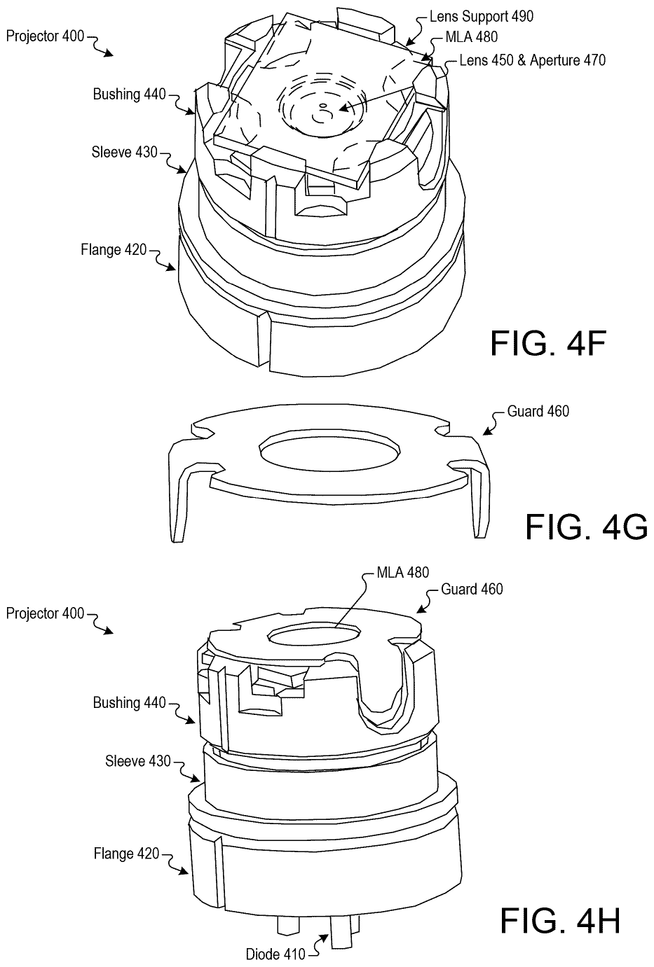

FIGS. 4A-H illustrate components of projectors 400 of intraoral scanners (e.g., intraoral scanner 300 of one or more of FIGS. 3A-N, scanner 150 of FIG. 1, and/or intraoral scanner 20 of one or more of FIGS. 2A-D), according to certain embodiments.

In some embodiments, intraoral scanner 300 includes one or more projectors. The projectors may be aligned and fixed in the intraoral scanner 300 (e.g., via projector alignment and fixing procedure). Each projector may provide alignment of a miniature optical lens to a laser source in three degrees of freedom (DOF) (e.g., x, y, and z) and may be robustly fixed by a welding process while avoiding potential movement caused by the weldment shrinkage. The projector may be a patterned projector aligned via active alignment (e.g., via laser welding, to constrain DOF). The mechanical hardware and alignment process of the projector may be designed to separate the DOFs to be aligned into two independent stages: X and Y (together); and Z. This separation allows to have no gaps between the mechanical items during the alignment process and may allow (e.g., guarantee) zero movement between the laser of the projector to the lens of the projector over the lifetime of the system (e.g., intraoral scanner 300). The projector may have a miniature welded structure. The projector may be fixed by welding (e.g., not by adhesive) for more robust, less thermally sensitive, and non-creep under stresses. The projector may have zero mechanical gap in the alignment and the welding process, such as no shrinkage forces (e.g., which would compromise the alignment result) during welding).

The housing of the projector may have three parts; flange; sleeve; and bushing. This may eliminate coupling between X-Y to Z axes and may maintain zero clearance between moving parts during alignment which may minimize undesired movement due to welding.

The sleeve may act as a mediator part which mechanically connects between the bushing (e.g., lens) and the flange (e.g., laser diode).

In the alignment process, the bushing is installed inside the sleeve internal diameter with no gap (e.g., no clearance) which allows the bushing to solely move in the z-direction relative to the sleeve. While the sleeve itself is constrained to connect with the flange with no gap, this allows the sleeve (e.g., and bushing together) to move only on the X-Y direction relative to the flange. While maintaining lens assembly and sleeve in aligned position relative to laser, at least three welding points may be applied simultaneously (e.g., equally spaced) around the axis. In some embodiments, lens assembly may be attached (e.g., welded) to the sleeve for fixing the z-axis (e.g., weld Z-DOF) and sleeve may be attached (e.g., welded) to flange for fixing X-Y axes (e.g., weld X-Y-DOF). This welding process may connect bushing to sleeve with no clearance; and sleeve to flange with no clearance. This zero-gap design and simultaneously 3x points welding may allow (e.g., guarantee) no movement occurs due to shrinkage of welding beads at each of the welding points. The alignment result may be kept while the welding is done and after.

FIG. 4A illustrates an upper perspective view of a projector 400. FIG. 4B illustrates an exploded view of a projector 400. FIG. 4C illustrates a lower perspective view of a projector 400. FIG. 4D illustrates a cross-sectional view of a projector 400. FIG. 4E illustrates a cross-sectional view of a projector 400.

Projector 400 includes one or more of diode 410, flange 420, sleeve 430, bushing 440, lens 450, aperture 470, micro lens array 480, and/or lens support 490.

Diode 410 (e.g., laser emitter) may provide (e.g., emit) a laser beam via a first distal end and may have connectors at a second distal end (e.g., two of which to be soldered to a PCB). Diode 410 may have tolerances (e.g., diameter datum and upper surface datum) that are greater than what is to be used for an intraocular scanner. Flange 420, sleeve 430, and bushing 440 may be used to constrain the diode 410 in the X-Y and Z directions. Flange 420 may secure the diode 410 (e.g., be disposed on diameter datum and upper surface datum of the diode 410). Flange 420 may secure the diode 410 prior to starting the alignment process. The flange 420 may be welded to the diode 410 (e.g., with at least four points of welding)

Bushing 440 (e.g., lens holder) may secure the lens 450, lens support, aperture 470, and/or MLA 480. Lens 450 may be an axicon collimator (e.g., to enhance the laser beam, shape the beam, put in precise position in x-direction and x-y-direction relative to the diode 410). Lens 450 may be a polymer lens or glass lens (e.g., glass lens with metal ring, metal ring may have apertures forming a recess for excess adhesive). Aperture 470 may be a pinhole aperture. Lens 450 and aperture 470 may be glued to the bushing 440 with adhesive (e.g., ultraviolet cured adhesive). Bushing 440 may be welded to the lens support 490 to secure the lens 450 and/or aperture 470 on the bushing 440.

MLA 480 may be a micro lens beam splitter array, beam splitter, array of micro lenses built on a glass substrate, diffractive optical element, diffractive element, transparent component above the diode 410, and/or the like. MLA 480 may divide a single beam into a multiple array of features (e.g., spots, dots). The microlens side of the MLA 480 may be proximate the aperture 470. Lens 450 may be secured by a metal ring.

Lens support 490 may secure the lens 450 and/or aperture 470 to the bushing 440. Aperture 470 may channel the laser beam from the diode 410. Lens 450 may collimate (e.g., align, make rays of light accurately parallel, etc.) the channeled laser beam. MLA 480 may cause the collimated and channeled laser beam become a projected pattern. The projected pattern may not be harmful to the eye.

Sleeve 430 (e.g., spacer) may be positioned around the bushing 440 and may abut flange 420. Sleeve 430 may be secured to bushing 440 at a particular height (e.g., z-direction) and may be secured to flange 420 at a particular X-Y position.

The diode 410 and flange 420 may be welded together (e.g., at four spots), lens support 490 and lens 450 may be connected with adhesive.

Lens support 490 secured to lens 450 may fit inside bushing 440 (e.g., in recesses formed by an upper surface of the bushing 440). X-Y direction may be lateral movement of the lens 450 relative to the laser beam. Projector 400 may control three degrees of freedom (e.g., x-direction, y-direction, and z-direction). The projector 400 may not be sensitive to rotation and/or the mechanical structure (e.g., flange 420, sleeve 430, and bushing 440) may provide accuracy of rotation (e.g., without alignment in the rotational degrees of freedom).

The sleeve 430 may first be secured to the bushing 440. This may be performed via a laser beam to an outer wall of sleeve 430 to bond the sleeve 430 to the bushing 440 (e.g., welded simultaneously in at least three points). This may control position of the projector 400 in the Z-direction (e.g., control height, align z-position with focal point).

Responsive to the sleeve 430 being secured to the bushing 440, the sleeve 430 may be secured (e.g., welded, laser welded, welded simultaneously in at least three points) to the flange 420 (e.g., control position in X-Y-direction). Sleeve 430 may be secured (e.g., via weld, via adhesive) to the flange 420 via a notch in the outer surface of the sleeve 430 and flange 420. In some embodiments, a welding procedure may build a weld bead in the notch. In some embodiments, a very thin adhesive may run capillary through notch to fill the area between the flange 420 and the sleeve 430.

In some embodiments, one or more of: the flange 420 may be secured to the diode 410 via adhesive; lens support 490 may be secured to bushing 440 via adhesive; sleeve 430 may be secured to bushing 440 via adhesive; and/or sleeve 430 may be secured to flange 420 via adhesive. The adhesive may be cured via heating.

The flange 420, sleeve 430, bushing 440, and lens support 490 may be the same type of material. The flange 420, sleeve 430, bushing 440, and lens support 490 may be the same type of metal. The metal may be steel (e.g., secured via adhesive), stainless steel (e.g., secured via laser welding), stainless steel 302, stainless steel 304, or aluminum (e.g., secured via laser welding). The flange 420, sleeve 430, bushing 440, and lens support 490 may be the same type of plastic (e.g., secured via laser welding, spot welding). The plastic may be glass-bead enforced plastic, carbon-enforced plastic, glass-reinforced nylon, or the like.

Components of the projector 400 may be used to align and fixate the lens 450 (e.g., axicollimator lens) and aperture 470 (e.g., that is center-bonded to lens 450) relative to the laser emission axis and point of diode 410 (e.g., using optical feedback). The components of the projector 400 may align and fixate the lens 450 and aperture 470 in the X, Y, and Z DOFs (e.g., axis being aligned) relative to laser emission axis (e.g., symmetry axis of the entire laser emission elliptical cone angle) and point using optical feedback from imaged laser beam quality and focus (e.g., symmetry and size of the laser beam image through the aperture 470 and lens 450). Components of the projector 400 may control laser emission point to lens distance, lens centration to laser axis, aperture centration to lens optical axis, lens tilt relative to leaser emission axis, and/or aperture tilt relative lens flat chamfer.

In some embodiments, the aperture 470 is glued to the lens 450 with centering under a microscope. The diode 410 (e.g., laser diode) may be welded to the flange 420. The lens support 490 is welded into bushing 440. Lens 450 with aperture 470 are glued into bushing 440. The sleeve 430 is welded to both flange 420 and bushing 440 under X, Y, and Z active alignment. The MLA 480 may be glued to the bushing 440.

The housing of the projector 400 may include three parts: flange 420; sleeve 430; and bushing 440. This may eliminate coupling between X-Y to Z axes and may maintain zero clearance (e.g., substantially zero clearance) between moving parts during alignment which may minimize undesired movement due to welding.

The diode 410 (e.g., diode case) may be welded to flange 420 (e.g., bottom of diode welded to bottom of flange 420. The lens support 490 may be welded into bushing 440 (e.g., lens support 490 welded to upper surface of bushing 440 at recesses formed by bushing 440). The lens may be centered (e.g., substantially centered) and/or glued to the aperture 470 (aperture 470 to be attached to planar side of lens 450). Aperture 470 may be centered (e.g., substantially centered) relative to the optical axis of the lens 450 (e.g., lens optical axis). The lens 450 and aperture 470 may be glued into bushing 440. Lens 450 and aperture 470 may be inserted from bottom fo bushing 440 with aperture 470 facing down.

The diode 410 (e.g., laser diode) and flange 420 (e.g., that have been welded together) may be attached to a fixed holder of a jig. The lens assembly (e.g., bushing 440, lens 450, aperture 470, lens support 490) and sleeve 430 may be attached to an X-Y-Z manipulation gripper. The bushing 440 may form a bushing feature (e.g., notch formed by outer surface) and flange 420 may form a flange feature (e.g., notch formed by outer surface) that are to be aligned during the welding of the bushing 440 and sleeve 430 to each other and the welding of the flange 420 and the sleeve 430 to each other. The X-Y-Z gripper may lower lens assembly and sleeve 430 in the z-direction until there is contact between flange 420 and sleeve 430 and between bushing 440 and sleeve 430. The lens assembly and sleeve 430 may be manipulated in the X, Y, and Z relative to the laser (e.g., diode 410) until spot quality criteria are met. Spot quality is to meet active alignment target quality criteria. The sleeve 430 is lowered in the z-axis to contact the flange 420.

While maintaining lens assembly and sleeve 430 in aligned position relative to the laser (e.g., diode 410), at least three substantially-equally spaced welding points are applied aper axis in the following order: lens assembly to sleeve 430 for fixing Z-axis; and sleeve 430 to flange 420 for fixing the X-Y axis. (See FIG. 4E.) The MLA 480 may be mounted onto the matching recess in the top of the lens assembly with lens profiles facing down (e.g., towards diode 410, towards laser). A controlled amount of adhesive may be applied to the corners (e.g., four corners) of the MLA 480 in the recesses (e.g., four recesses).

FIG. 4F illustrates a perspective view of a projector 400. FIG. 4G illustrates a guard 460 (e.g., MLA guard) of a projector 400. FIG. 4H illustrates a perspective view of a projector 400.

The MLA 480 (e.g., lens array) may receive a single laser beam and divide the single laser beam into multiple laser points (e.g., multiple laser beams). The single laser beam may be dangerous to eyes (e.g., eye safety hazard, single beam energy concentration is very high for eye exposure). The multiple laser points may be safe for eyes (e.g., reduce density of each of the single beams, not hazardous to eyes). MLA 480 may be placed on and glued (e.g., with ultraviolet (UV) adhesive at four points) to the bushing 440 in recesses formed by the upper surface of the bushing 440. If the MLA 480 were not glued correctly, the MLA 480 could fall and could cause an eye safety hazard.

The guard 460 may be used in addition to or instead of gluing the MLA 480 to the bushing 440. The guard 460 may have at least two projections that interface with corresponding recesses (e.g., at least two recesses) formed by the outer surface of the bushing 440. The at least two projections of the guard 460 may be welded to the bushing 440 at the corresponding recesses formed by the bushing 440. The MLA 480 glued to the bushing 440 and the guard 460 welded to the bushing 440 provides a double fault solution. If the MLA 480 were to detach from the bushing 440, it would be trapped inside the projector 400 by the guard 460. The guard 460 may be stainless steel sheet metal that is folded to form the projections. In some embodiments, the guard 460 is welded to the sides of the bushing 440. In some embodiments, the guard 460 is welded to the top of the bushing 440.

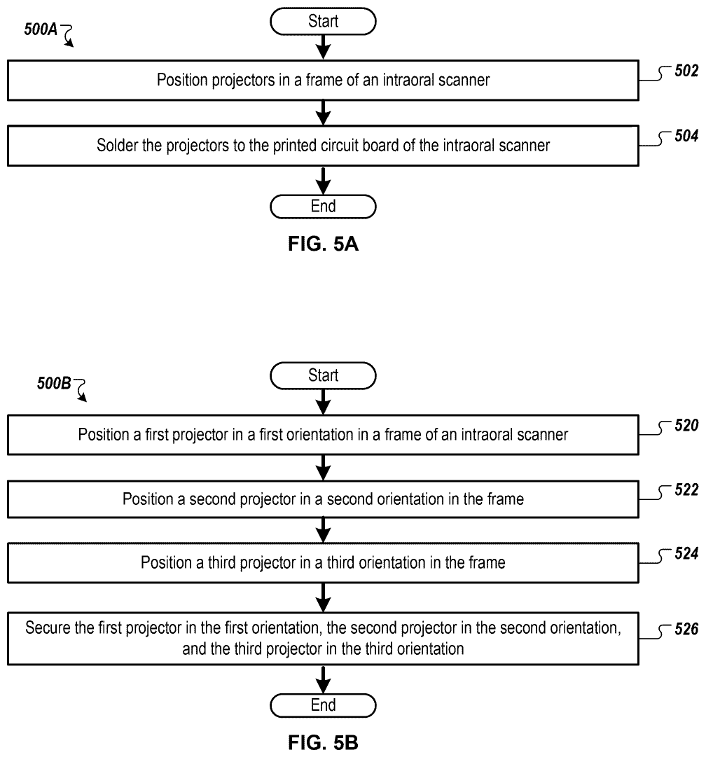

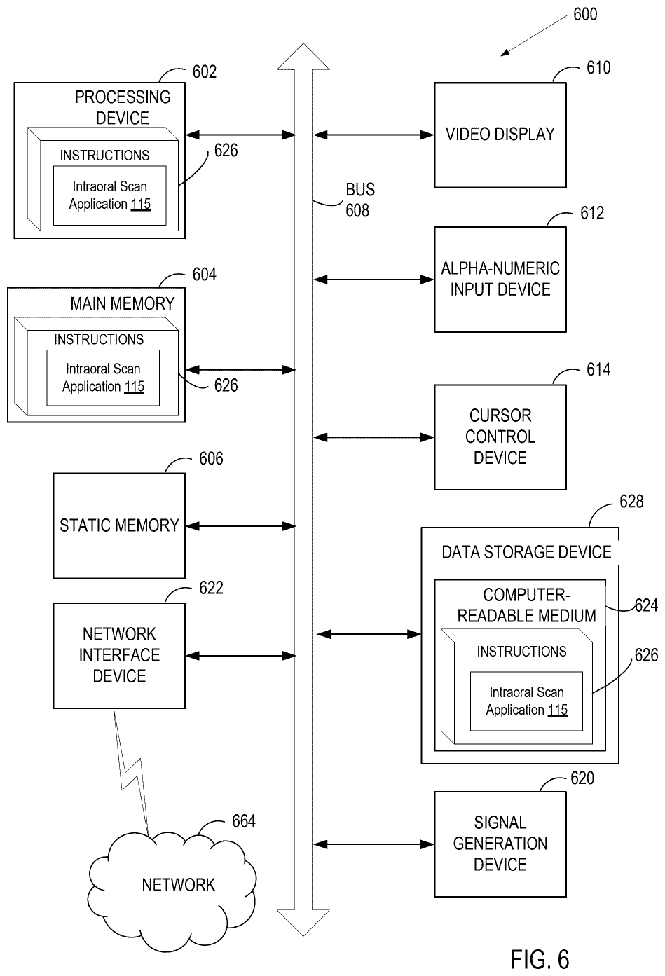

FIGS. 5A-B are flow charts of methods associated with projectors of intraoral scanners, according to certain embodiments. The methods may be performed by a processing logic that may include hardware (e.g., circuitry, dedicated logic, programmable logic, microcode, etc.), software (e.g., instructions run on a processing device to perform hardware simulation), firmware, or a combination thereof. In some embodiments, at least some operations of the methods are performed by a computing device of a scanning system and/or by a server computing device (e.g., by computing device 105 of FIG. 1 or computing device 600 of FIG. 6).

Components described with respect to one or more of FIGS. 1-4H may be used to illustrate aspects of FIGS. 5A-B. In some embodiments, a non-transitory machine-readable storage medium stores instructions that when executed by a processing device (e.g., of computing device 105 of FIG. 1, computing device 600 of FIG. 6, etc.) cause the processing device to perform methods 500A-B.

For simplicity of explanation, methods 500A-B are depicted and described as a series of acts. However, acts in accordance with this disclosure can occur in various orders and/or concurrently and with other acts not presented and described herein. Furthermore, not all illustrated acts may be performed to implement the methods 500A-B in accordance with the disclosed subject matter. In addition, those skilled in the art will understand and appreciate that the methods 500A-B could alternatively be represented as a series of interrelated states via a state diagram or events.

FIG. 5A is a flow chart for a method 500A associated with positioning projectors in an intraoral scanner, according to certain embodiments.

At block 502 of method 500A, projectors are positioned (e.g., by processing logic, manually, etc.) in a frame of an intraoral scanner.

At block 504, the projectors are soldered (e.g., by processing logic, manually, etc.) to the printed circuit board (PCB) of the intraoral scanner. In some embodiments, block 504 is responsive to block 502. In some embodiments, the intraoral scanner is configured to provide intraoral scans of a dental site during an intraoral scanning session. In some embodiments, the intraoral scans are generated by projecting, via the projectors, a structured light including features (e.g., spots) onto the dental site and capturing the features on the dental site. Responsive to block 504, the projectors may be secured to the frame via curing adhesive. The projectors may be secured to a frame (e.g., projector frame) that is separate from a camera frame that secures the cameras.

FIG. 5B is a flow chart for a method 500B associated with positioning projectors in an intraoral scanner, according to certain embodiments.

At block 520 of method 500B, a first projector is positioned (e.g., by processing logic, manually, etc.) in a first orientation in a frame of an intraoral scanner.

At block 522, a second projector is positioned (e.g., by processing logic, manually, etc.) in a second orientation in the frame. In some embodiments, the second orientation is rotated about 3 to about 28 degrees from the first orientation.

At block 524, a third projector is positioned (e.g., by processing logic, manually, etc.) in a third orientation in the frame. In some embodiments, the third orientation is rotated about 3 to about 28 degrees from the second orientation.

At block 526, the first projector in the first orientation, second projector in the second orientation, and the third projector in the third orientation are secured (e.g., by processing logic, manually, etc.) in the intraoral scanner. In some embodiments, the first projector in the first orientation, second projector in the second orientation, and the third projector in the third orientation being secured reduces ambiguity of scanning of a dental site via the intraoral scanner. In some embodiments, block 526 is via soldering the first projector, the second projector, and the third projector to a PCB. In some embodiments, responsive to soldering the projectors to the PCB, the projectors are secured to the frame via curing of adhesive between the frame and each projector.

In some embodiments, three or more projectors are oriented and secured in the intraoral scanner. In some embodiments, each of the projectors are disposed in a different orientation in the intraoral scanner. For example, a first projector may be oriented at about −10 degrees, a second projector may be oriented at about −5 degrees, a third projector may be oriented at about 0 degrees, a fourth projector may be oriented at about +5 degrees, and a fifth projector may be oriented at about +10 degrees.

FIG. 6 illustrates a block diagram of an example computing device 600, according to certain embodiments. In some embodiments, FIG. 6 illustrates a diagrammatic representation of a machine in the example form of a computing device 600 within which a set of instructions, for causing the machine to perform any one or more of the methodologies discussed herein, may be executed. In some embodiments, the machine may be connected (e.g., networked) to other machines in a Local Area Network (LAN), an intranet, an extranet, or the Internet. The computing device 600 may correspond, for example, to computing device 105 and/or computing device 106 of FIG. 1. The machine may operate in the capacity of a server or a client machine in a client-server network environment, or as a peer machine in a peer-to-peer (or distributed) network environment. The machine may be a personal computer (PC), a tablet computer, a set-top box (STB), a Personal Digital Assistant (PDA), a cellular telephone, a web appliance, a server, a network router, switch or bridge, or any machine capable of executing a set of instructions (sequential or otherwise) that specify actions to be taken by that machine. Further, while only a single machine is illustrated, the term “machine” shall also be taken to include any collection of machines (e.g., computers) that individually or jointly execute a set (or multiple sets) of instructions to perform any one or more of the methodologies discussed herein.

The example computing device 600 includes a processing device 602, a main memory 604 (e.g., read-only memory (ROM), flash memory, dynamic random access memory (DRAM) such as synchronous DRAM (SDRAM), etc.), a static memory 606 (e.g., flash memory, static random access memory (SRAM), etc.), and a secondary memory (e.g., a data storage device 628), which communicate with each other via a bus 608.

Processing device 602 represents one or more general-purpose processors such as a microprocessor, central processing unit, or the like. More particularly, the processing device 602 may be a complex instruction set computing (CISC) microprocessor, reduced instruction set computing (RISC) microprocessor, very long instruction word (VLIW) microprocessor, processor implementing other instruction sets, or processors implementing a combination of instruction sets. Processing device 602 may also be one or more special-purpose processing devices such as an application specific integrated circuit (ASIC), a field programmable gate array (FPGA), a digital signal processor (DSP), network processor, or the like. Processing device 602 is configured to execute the processing logic (instructions 626) for performing operations and steps discussed herein.

The computing device 600 may further include a network interface device 622 for communicating with a network 664. The computing device 600 also may include a video display unit 610 (e.g., a liquid crystal display (LCD) or a cathode ray tube (CRT)), an alphanumeric input device 612 (e.g., a keyboard), a cursor control device 614 (e.g., a mouse), and a signal generation device 620 (e.g., a speaker).

The data storage device 628 may include a machine-readable storage medium (or more specifically a non-transitory computer-readable storage medium) 624 on which is stored one or more sets of instructions 626 embodying any one or more of the methodologies or functions described herein, such as instructions for intraoral scan application 615, which may correspond to intraoral scan application 115 of FIG. 1. A non-transitory storage medium refers to a storage medium other than a carrier wave. The instructions 626 may also reside, completely or at least partially, within the main memory 604 and/or within the processing device 602 during execution thereof by the computing device 600, the main memory 604 and the processing device 602 also constituting computer-readable storage media.

The computer-readable storage medium 624 may also be used to store intraoral scan application 115, which may include one or more machine learning modules, and which may perform the operations described herein above. The computer readable storage medium 624 may also store a software library containing methods for the intraoral scan application 115. While the computer-readable storage medium 624 is shown in an example embodiment to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any medium other than a carrier wave that is capable of storing or encoding a set of instructions for execution by the machine and that cause the machine to perform any one or more of the methodologies of the present disclosure. The term “computer-readable storage medium” shall accordingly be taken to include, but not be limited to, solid-state memories, and optical and magnetic media.

In some embodiments, the methods, components, and features described herein are implemented by discrete hardware components or are integrated in the functionality of other hardware components such as ASICS, FPGAs, DSPs, or similar devices. In some embodiments, the methods, components, and features are implemented by firmware modules or functional circuitry within hardware devices. In some embodiments, the methods, components, and features are implemented in any combination of hardware devices and computer program components, or in computer programs.

Unless specifically stated otherwise, terms such as “transmitting,” “receiving,” “identifying,” “determining,” “generating,” “providing,” “obtaining,” “causing,” or the like, refer to actions and processes performed or implemented by computer systems that manipulates and transforms data represented as physical (electronic) quantities within the computer system registers and memories into other data similarly represented as physical quantities within the computer system memories or registers or other such information storage, transmission or display devices. In some embodiments, the terms “first,” “second,” “third,” “fourth,” etc. as used herein are meant as labels to distinguish among different elements and do not have an ordinal meaning according to their numerical designation.

Examples described herein also relate to an apparatus for performing the methods described herein. In some embodiments, this apparatus is specially constructed for performing the methods described herein or includes a general-purpose computer system selectively programmed by a computer program stored in the computer system. Such a computer program is stored in a computer-readable tangible storage medium.

Some of the methods and illustrative examples described herein are not inherently related to any particular computer or other apparatus. In some embodiments, various general-purpose systems are used in accordance with the teachings described herein. In some embodiments, a more specialized apparatus is constructed to perform methods described herein and/or each of their individual functions, routines, subroutines, or operations. Examples of the structure for a variety of these systems are set forth in the description above.

The above description is intended to be illustrative, and not restrictive. Although the present disclosure has been described with references to specific illustrative examples and implementations, it will be recognized that the present disclosure is not limited to the examples and implementations described. The scope of the disclosure should be determined with reference to the following claims, along with the full scope of equivalents to which the claims are entitled.

The preceding description sets forth numerous specific details such as examples of specific systems, components, methods, and so forth in order to provide a good understanding of several embodiments of the present disclosure. It will be apparent to one skilled in the art, however, that at least some embodiments of the present disclosure may be practiced without these specific details. In other instances, well-known components or methods are not described in detail or are presented in simple block diagram format in order to avoid unnecessarily obscuring the present disclosure. Thus, the specific details set forth are merely exemplary. Particular implementations may vary from these exemplary details and still be contemplated to be within the scope of the present disclosure.

The terms “over,” “under,” “between,” “disposed on,” and “on” as used herein refer to a relative position of one material layer or component with respect to other layers or components. For example, one layer disposed on, over, or under another layer may be directly in contact with the other layer or may have one or more intervening layers. Moreover, one layer disposed between two layers may be directly in contact with the two layers or may have one or more intervening layers. Similarly, unless explicitly stated otherwise, one feature disposed between two features may be in direct contact with the adjacent features or may have one or more intervening layers.

The words “example” or “exemplary” are used herein to mean serving as an example, instance or illustration. Any aspect or design described herein as “example’ or “exemplary” is not necessarily to be construed as preferred or advantageous over other aspects or designs. Rather, use of the words “example” or “exemplary” is intended to present concepts in a concrete fashion.

Reference throughout this specification to “one embodiment,” “an embodiment,” or “some embodiments” means that a particular feature, structure, or characteristic described in connection with the embodiment is included in at least one embodiment. Thus, the appearances of the phrase “in one embodiment,” “in an embodiment,” or “in some embodiments” in various places throughout this specification are not necessarily all referring to the same embodiment. In addition, the term “or” is intended to mean an inclusive “or” rather than an exclusive “or.” That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Also, the terms “first,” “second,” “third,” “fourth,” etc. as used herein are meant as labels to distinguish among different elements and can not necessarily have an ordinal meaning according to their numerical designation. When the term “about,” “substantially,” or “approximately” is used herein, this is intended to mean that the nominal value presented is precise within ±10%.

Although the operations of the methods herein are shown and described in a particular order, the order of operations of each method may be altered so that certain operations may be performed in an inverse order so that certain operations may be performed, at least in part, concurrently with other operations. In another embodiment, instructions or sub-operations of distinct operations may be in an intermittent and/or alternating manner.

It is understood that the above description is intended to be illustrative, and not restrictive. Many other embodiments will be apparent to those of skill in the art upon reading and understanding the above description. The scope of the disclosure should, therefore, be determined with reference to the appended claims, along with the full scope of equivalents to which such claims are entitled.

What is claimed is:

1. An intraoral scanner comprising:
a rigid structure coupling with a plurality of structured light projectors and a plurality of cameras, wherein:
a corresponding field of view of each camera of the plurality of cameras overlaps a respective field of view of a corresponding neighboring camera of the plurality of cameras,
the corresponding field of view of each camera of the plurality of cameras overlaps a corresponding field of illumination of a corresponding neighboring structured light projector of the plurality of structured light projectors;
a structured light projector of the plurality of structured light projectors is secured to a metallic frame at an opening of the metallic frame;
each structured light projector of the plurality of structured light projectors comprises: a light source configured to generate light; one or more projector lenses to receive the light generated from the light source; one or more projector lens frames configured to secure the one or more projector lenses relative to the light source; and a pattern generating optical element configured to receive the light and generate a pattern of light along a projector axis onto an intraoral surface; and
each of the plurality of cameras comprises: an image sensor configured to capture a plurality of images along an optical axis that depict at least a portion of the pattern of light projected on the intraoral surface by the corresponding neighboring structured light projector; and one or more camera lenses.

2. The intraoral scanner of claim 1, wherein the structured light projector is attached to the metallic frame by a cured adhesive.

3. The intraoral scanner of claim 2, wherein the cured adhesive is a thermally conductive adhesive that improves thermal connection of the structured light projector to the metallic frame.

4. The intraoral scanner of claim 1, wherein the rigid structure is configured to maintain stable structural integrity and positioning of the plurality of structured light projectors and the plurality of cameras with respect to each other.

5. The intraoral scanner of claim 1, wherein the rigid structure is configured to maintain geometric integrity of optics of each of the plurality of structured light projectors and each of the plurality of cameras under varying ambient conditions.

6. The intraoral scanner of claim 1, wherein the corresponding field of view of each camera of the plurality of cameras overlaps with at least 50% of a pattern projected by the corresponding neighboring structured light projector at an object focal plane that is located at least 4 mm from a camera lens that is farthest from a camera sensor.

7. The intraoral scanner of claim 6, wherein the corresponding field of view of each camera of the plurality of cameras overlaps with at least 75% of the pattern projected by the corresponding neighboring structured light projector at the object focal plane that is located at least 4 mm from the camera lens that is farthest from the camera sensor.

8. The intraoral scanner of claim 7, wherein the plurality of cameras are configured to capture the plurality of images at a frame rate of at least 30 frames per second.

9. The intraoral scanner of claim 8, wherein the plurality of cameras are configured to capture the plurality of images at the frame rate of at least 75 frames per second.

10. The intraoral scanner of claim 1, wherein each of the plurality of structured light projectors is configured to collimate the light from the light source.

11. The intraoral scanner of claim 10, wherein each of the plurality of structured light projectors is configured to collimate the light from the light source using a lens.

12. The intraoral scanner of claim 1, wherein optical axes of adjacent cameras of the plurality of cameras are non-parallel.

13. The intraoral scanner of claim 12, wherein an angle between the optical axes of the adjacent cameras is 90 degrees or less.

14. The intraoral scanner of claim 13, wherein the angle between the optical axes of the adjacent cameras is 35 degrees or less.

15. The intraoral scanner of claim 1, wherein the structured light projector is positioned within the metallic frame via a flange.

16. An intraoral scanner comprising:
a means for coupling with a plurality of structured light projectors and a plurality of cameras; and
a means for securing a structured light projector of the plurality of structured light projectors at an opening of the means for securing the structured light projector, wherein:
a corresponding field of view of each camera of the plurality of cameras overlaps a respective field of view of a corresponding neighboring camera of the plurality of cameras;
the corresponding field of view of each camera of the plurality of cameras overlaps a corresponding field of illumination of a corresponding neighboring structured light projector of the plurality of structured light projectors;
each structured light projector of the plurality of structured light projectors comprises: a light source configured to generate light; one or more projector lenses to receive the light generated from the light source; one or more projector lens frames configured to secure the one or more projector lenses relative to the light source; and a pattern generating optical element configured to receive the light and generate a pattern of light along a projector axis onto an intraoral surface; and
each of the plurality of cameras comprises: an image sensor configured to capture a plurality of images along an optical axis that depict at least a portion of the pattern of light projected on the intraoral surface by the corresponding neighboring structured light projector; and one or more camera lenses.

17. The intraoral scanner of claim 16 further comprising a means for securing the plurality of cameras.

18. The intraoral scanner of claim 16, wherein the corresponding field of view of each camera of the plurality of cameras overlaps with at least 50% of a pattern projected by the corresponding neighboring structured light projector at an object focal plane that is located at least 4 mm from a camera lens that is farthest from a camera sensor.

19. The intraoral scanner of claim 18, wherein the corresponding field of view of each camera of the plurality of cameras overlaps with at least 75% of the pattern projected by the corresponding neighboring structured light projector at the object focal plane that is located at least 4 mm from the camera lens that is farthest from the camera sensor.

20. A method of manufacturing an intraoral scanner, the method comprising:
coupling a plurality of structured light projectors and a plurality of cameras to a rigid structure so that a corresponding field of view of each camera of the plurality of cameras overlaps a respective field of view of a corresponding neighboring camera of the plurality of cameras and so that the corresponding field of view of each camera of the plurality of cameras overlaps a corresponding field of illumination of a corresponding neighboring structured light projector of the plurality of structured light projectors, wherein:
coupling the plurality of structured light projectors to the rigid structure comprises securing a structured light projector of the plurality of structured light projectors to a metallic frame by positioning the structured light projector into a dedicated projector opening of the metallic frame with adhesive and attaching the structured light projector to the metallic frame by curing the adhesive;
each structured light projector of the plurality of structured light projectors comprises: a light source configured to generate light; one or more projector lenses to receive the light generated from the light source; one or more projector lens frames configured to secure the one or more projector lenses relative to the light source; and a pattern generating optical element configured to receive the light and generate a pattern of light along a projector axis onto an intraoral surface; and
each of the plurality of cameras comprises: an image sensor configured to capture a plurality of images along an optical axis that depict at least a portion of the pattern of light projected on the intraoral surface by the corresponding neighboring structured light projector; and one or more camera lenses.

21. The method of claim 20, wherein the adhesive is a thermally conductive adhesive that improves thermal connection of the structured light projector and the metallic frame.

22. The method of claim 20, wherein the structured light projector fits within the dedicated projector opening of the metallic frame without an air gap to cause heat to be dissipated from the structured light projector through the metallic frame.

23. The method of claim 20, wherein coupling the plurality of cameras to the rigid structure comprises securing the plurality of cameras with the metallic frame.

24. The method of claim 23, wherein securing the plurality of cameras with the metallic frame comprises securing the plurality of cameras with a camera frame.

25. The method of claim 24, wherein the metallic frame is a projector frame, wherein the camera frame and the projector frame are separate components, and wherein securing the plurality of cameras with the metallic frame further comprises securing the camera frame with the projector frame.

26. The method of claim 24, wherein the metallic frame is a projector frame, wherein the camera frame and the projector frame are not separate components.

27. The method of claim 20, wherein the plurality of structured light projectors and the plurality of cameras are coupled to the rigid structure such that the corresponding field of view of each camera of the plurality of cameras overlaps with at least 50% of a pattern projected by the corresponding neighboring structured light projector at an object focal plane that is located at least 4 mm from a camera lens that is farthest from a camera sensor.

28. The method of claim 27, wherein the plurality of structured light projectors and the plurality of cameras are coupled to the rigid structure such that the corresponding field of view of each camera of the plurality of cameras overlaps with at least 75% of the pattern projected by the corresponding neighboring structured light projector at the object focal plane that is located at least 4 mm from the camera lens that is farthest from the camera sensor.
